(19) United States
(12) Patent Application Publication
(10) Pub. No.: US 2006/0062761 A1
(43) Pub. Date: Mar. 23, 2006

(54) Modified interferon alpha with reduced immunogenicity

(76) Inventors: Francis J. Carr, Aberdeenshire (GB); Graham Carter, Abendeenshire (GB); Tim Jones, Cambridge (GB); Matthew Baker, Cambridge (GB); John Watkins, Cambridge (GB); Marian Hanlon, Cambridge (GB)

Correspondence Address: OLSON & HIERL, LTD., 20 NORTH WACKER DRIVE, 36TH FLOOR, CHICAGO, IL, 60606, US

(21) Appl. No.: 10/469,679

(22) PCT Filed: Mar. 1, 2002
(86) PCT No.: PCT/EP02/02218

§ 371 Date: Sep. 2, 2003

(30) Foreign Application Priority Data
Mar. 2, 2001 (EP) 01105088.7

(51) Int. Cl. C07K 14/56; C07H 21/04; C12P 21/04; A61K 38/21

(52) U.S. Cl. 424/85.7; 530/351; 435/69.51; 435/320.1; 435/325; 536/23.5

(57) ABSTRACT

The present invention relates to polypeptides to be administered especially to humans and in particular for therapeutic use. The polypeptides are modified polypeptides whereby the modification results in a reduced propensity for the polypeptide to elicit an immune response upon administration to the human subject. The invention in particular to the modification of human interferon alpha and specifically interferon alpha 2(INFα2) to result in proteins that are substantially non-immunogenic or less immunogenic than any non-modified counterpart when use in vivo.

### FIELD OF THE INVENTION

The present invention relates to polypeptides to be administered especially to humans and in particular for therapeutic use. The polypeptides are modified polypeptides whereby the modification results in a reduced propensity for the polypeptide to elicit an immune response upon administration to the human subject. The invention in particular relates to the modification of human interferon and specifically human interferon α2 (INFα2) to result in INFα2 protein variants that are substantially non-immunogenic or less immunogenic than any non-modified counterpart when used in vivo. The invention relates furthermore to T-cell epitope peptides derived from said non-modified protein by means of which it is possible to create modified INFα2 variants with reduced immunogenicity.

### BACKGROUND OF THE INVENTION

There are many instances whereby the efficacy of a therapeutic protein is limited by an unwanted immune reaction to the therapeutic protein. Several mouse monoclonal antibodies have shown promise as therapies in a number of human disease settings but in certain cases have failed due to the induction of significant degrees of a human anti-murine antibody (HAMA) response [Schroff, R. W. et al (1985) Cancer Res. 45: 879-885; Shawler, D. L. et al (1985) J. Immunol. 135: 1530-1535]. For monoclonal antibodies, a number of techniques have been developed in attempt to reduce the HAMA response [WO 89/09622; EP 0239400; EP 0438310; WO 91/06667]. These recombinant DNA approaches have generally reduced the mouse genetic information in the final antibody construct whilst increasing the human genetic information in the final construct. Notwithstanding, the resultant “humanized” antibodies have, in several cases, still elicited an immune response in patients [Issacs J. D. (1990) Sem. Immunol. 2: 449, 456; Rebello, P. R. et al (1999) Transplantation 68: 1417-1420].

Antibodies are not the only class of polypeptide molecule administered as a therapeutic agent against which an immune response may be mounted. Even proteins of human origin and with the same amino acid sequences as occur within humans can still induce an immune response in humans. Notable examples include the therapeutic use of granulocyte-macrophage colony stimulating factor [Wadhwa, M. et al (1999) Clin. Cancer Res. 5: 1353-1361] and INFα2 [Russo, D. et al (1996) Bri. J. Haem. 94: 300-305; Stein, R. et al (1988) New Engl. J. Med. 318: 1409-1413] amongst others.

A principal factor in the induction of an immune response is the presence within the protein of peptides that can stimulate the activity of T-cells via presentation on MHC class II molecules, so-called “T-cell epitopes”. Such potential T-cell epitopes are commonly defined as any amino acid residue sequence with the ability to bind to MHC Class II molecules. Such T-cell epitopes can be measured to establish MHC binding. Implicitly, a “T-cell epitope” means an epitope which when bound to MHC molecules can be recognized by a T-cell receptor (TCR), and which can, at least in principle, cause the activation of these T-cells by engaging a TCR to promote a T-cell response. It is, however, usually understood that certain peptides which are found to bind to MHC Class II molecules may be retained in a protein sequence because such peptides are recognized as “self” within the organism into which the final protein is administered.

It is known, that certain of these T-cell epitope peptides can be released during the degradation of peptides, polypeptides or proteins within cells and subsequently be presented by molecules of the major histocompatability complex (MHC) in order to trigger the activation of T-cells. For peptides presented by MHC Class II, such activation of T-cells can then give rise, for example, to an antibody response by direct stimulation of B-cells to produce such antibodies.

MHC Class II molecules are a group of highly polymorphic proteins which play a central role in helper T-cell selection and activation. The human leukocyte antigen group DR (HLA-DR) are the predominant isotype of this group of proteins and are the major focus of the present invention. However, isotypes HLA-DQ and HLA-DP perform similar functions, hence the present invention is equally applicable to these. The MHC class II DR molecule is made of an alpha and a beta chain which insert at their C-termini through the cell membrane. Each hetero-dimer possesses a ligand binding domain which binds to peptides varying between 9 and 20 amino acids in length, although the binding groove can accommodate a maximum of 11 amino acids. The ligand binding domain is comprised of amino acids 1 to 85 of the alpha chain, and amino acids 1 to 94 of the beta chain. DQ molecules have recently been shown to have an homologous structure and the DP family proteins are also expected to be very similar. In humans approximately 70 different allotypes of the DR isotype are known, for DQ there are 30 different allotypes and for DP 47 different allotypes are known. Each individual bears two to four DR alleles, two DQ and two DP alleles. The structure of a number of DR molecules has been solved and such structures point to an open-ended peptide binding groove with a number of hydrophobic pockets which engage hydrophobic residues (pocket residues) of the peptide [Brown et al Nature (1993) 364: 33; Stern et al (1994) Nature 368: 215]. Polymorphism identifying the different allotypes of class II molecule contributes to a wide diversity of different binding surfaces for peptides within the peptide binding grove and at the population level ensures maximal flexibility with regard to the ability to recognize foreign proteins and mount an immune response to pathogenic organisms. There is a considerable amount of polymorphism within the ligand binding domain with distinct “families” within different geographical populations and ethnic groups. This polymorphism affects the binding characteristics of the peptide binding domain, thus different “families” of DR molecules will have specificities for peptides with different sequence properties, although there may be some overlap. This specificity determines recognition of Th-cell epitopes (Class II T-cell response) which are ultimately responsible for driving the antibody response to B-cell epitopes present on the same protein from which the Th-cell epitope is derived. Thus, the immune response to a protein in an individual is heavily influenced by T-cell epitope recognition which is a function of the peptide binding specificity of that individual's HLA-DR allotype. Therefore, in order to identify T-cell epitopes within a protein or peptide in the context of a global population, it is desirable to consider the binding properties of as diverse a set of HLA-DR allotypes as possible, thus covering as high a percentage of the world population as possible.

An immune response to a therapeutic protein such as the protein which is object of this invention, proceeds via the MHC class II peptide presentation pathway. Here exogenous proteins are engulfed and processed for presentation in association with MHC class II molecules of the DR, DQ or DP type. MHC Class II molecules are expressed by professional antigen presenting cells (APCs), such as macrophages and dendritic cells amongst others. Engagement of a MHC class II peptide complex by a cognate T-cell receptor on the surface of the T-cell, together with the cross-binding of certain other co-receptors such as the CD4 molecule, can induce an activated state within the T-cell.

Activation leads to the release of cytokines further activating other lymphocytes such as B cells to produce antibodies or activating T killer cells as a full cellular immune response. The ability of a peptide to bind a given MHC class II molecule for presentation on the surface of an APC is dependent on a number of factors most notably its primary sequence. This will influence both its propensity for proteolytic cleavage and also its affinity for binding within the peptide binding cleft of the MHC class II molecule. The MHC class II/peptide complex on the APC surface presents a binding face to a particular T-cell receptor (TCR) able to recognize determinants provided both by exposed residues of the peptide and the MHC class II molecule.

In the art there are procedures for identifying synthetic peptides able to bind MHC class II molecules (e.g. WO98/52976 and WO00/34317). Such peptides may not function as T-cell epitopes in all situations, particularly, in vivo due to the processing pathways or other phenomena. T-cell epitope identification is the first step to epitope elimination. The identification and removal of potential T-cell epitopes from proteins has been previously disclosed. In the art methods have been provided to enable the detection of T-cell epitopes usually by computational means scanning for recognized sequence motifs in experimentally determined T-cell epitopes or alternatively using computational techniques to predict MHC class II-binding peptides and in particular DR-binding peptides.

WO98/52976 and WO00/34317 teach computational threading approaches to identifying polypeptide sequences with the potential to bind a sub-set of human MHC class II DR allotypes. In these teachings, predicted T-cell epitopes are removed by the use of judicious amino acid substitution within the primary sequence of the therapeutic antibody or non-antibody protein of both non-human and human derivation.

Other techniques exploiting soluble complexes of recombinant MHC molecules in combination with synthetic peptides and able to bind to T-cell clones from peripheral blood samples from human or experimental animal subjects have been used in the art [Kern, F. et al (1998) Nature Medicine 4:975-978; Kwok, W. W. et al (2001) TRENDS in Immunology 22: 583-588] and may also be exploited in an epitope identification strategy.

As depicted above and as consequence thereof, it would be desirable to identify and to remove or at least to reduce T-cell epitopes from a given in principal therapeutically valuable but originally immunogenic peptide, polypeptide or protein.

One of these therapeutically valuable molecules is INFα2. The molecule is an important glycoprotein cytokine expressed by activated macrophages. The protein has antiviral activity and stimulates the production of at least two enzymes; a protein kinase and an oligoadenylate synthetase, on binding to the interferon alpha receptor in expressing cells. The mature INFα2 protein is single polypeptide of 165 amino acids produced by post-translational processing of a 188 amino acid pre-cursor protein by cleavage of a 23 amino acid signal sequence from the amino terminus. Several different subtypes of human INFα2 are known showing minor differences between primary amino acid sequences. Thus INFα2a and INFα2b differ in only one residue at position 23 of the mature protein chain being lysine in INFα2a and arginine in INFα2b. Whilst the disclosures of the present invention are directed towards the sequence of INFα2b, it can be seen that for all practical purposes the sequence of INFα2a may be considered interchangeably with the subject INFα2b subtype of the present invention. The amino acid sequence of INFα2(a,b) (depicted as one-letter code) is as follows:

CDLPQTHSLGSRRTLMLLAQMR (R,K) ISLFSCLKDRHDFGFPQEEFG
NQFQKAETIPVLHEMIQQIFNLFSTKDSSAAWDETLLDKFYTELYQQLND
LEACVIQGVGVTETPLMKEDSILAVRKYFQRITLYLKEKKYSPCAWEVVR
AEIMRSFSLSTNLQESLRSKE

The protein has considerable clinical importance as a broad spectrum anti-viral, anti-proliferative and immunomodulating agent. Recombinant and other preparations of INFα2 have been used therapeutically in a variety of cancer and viral indications in man [reviewed in Sen, G. G. and Lengyel P, (1992), J. Biol. Chem. 267: 5017-5020]. However despite very significant therapeutic benefit to large numbers of patients, resistance to therapy in certain patients has been documented and one important mechanism of resistance has been shown to be the development of neutralising antibodies detectable in the serum of treated patients [Quesada, J. R. et al (1985) J. Clin. Oncology 3:1522-1528; Stein R. G. et al (1988) ibid; Russo, D. et al (1996) ibid; Brooks M. G. et al (1989) Gut 30: 1116-1122]. An immune response in these patients is mounted to the therapeutic interferon despite the fact that a molecule of at least identical primary structure is produced endogenously in man.

Others have provided modified INFα2a and methods of use [U.S. Pat. No. 4,496,537; U.S. Pat. No. 5,972,331; U.S. Pat. No. 5,480,640; U.S. Pat. No. 5,190,751; U.S. Pat. No. 4,959,210], but these approaches have been directed towards improvements in the commercial production of INFα2a. Such teachings do not recognize the importance of T-cell epitopes to the immunogenic properties of the protein nor have been conceived to directly influence said properties in a specific and controlled way according to the scheme of the present invention.

However, there is a continued need for INFα2a analogues with enhanced properties. Desired enhancements include alternative schemes and modalities for the expression and purification of the said therapeutic, but also and especially, improvements in the biological properties of the protein. There is a particular need for enhancement of the in vivo characteristics when administered to the human subject. In this regard, it is highly desired to provide INFα2a with reduced or absent potential to induce an immune response in the human subject.

### SUMMARY AND DESCRIPTION OF THE INVENTION

The present invention provides for modified forms of human interferon α, and specifically the interferon α2 type, herein called “INFα2”, in which the immune characteristic is modified by means of reduced or removed numbers of potential T-cell epitopes.

The invention discloses sequences identified within the INFα2 primary sequence that are potential T-cell epitopes by virtue of MHC class II binding potential. This disclosure specifically pertains the human INFα2 protein being 165 amino acid residues. The invention discloses also specific positions within the primary sequence of the molecule which according to the invention are to be altered by specific amino acid substitution, addition or deletion without in principal affecting the biological activity. In cases in which the loss of immunogenicity can be achieved only by a simultaneous loss of biological activity it is possible to restore said activity by further alterations within the amino acid sequence of the protein.

The invention furthermore discloses methods to produce such modified molecules, and above all methods to identify said T-cell epitopes which require alteration in order to reduce or remove immunogenic sites.

The protein according to this invention would expect to display an increased circulation time within the human subject and would be of particular benefit in chronic or recurring disease settings such as is the case for a number of indications for INFα2. The present invention provides for modified forms of INFα2 proteins that are expected to display enhanced properties in vivo. These modified INFα2 molecules can be used in pharmaceutical compositions.

In summary the invention relates to the following issues:
a modified molecule having the biological activity of human interferon alpha 2 (INFα2) and being substantially non-immunogenic or less immunogenic than any non-modified molecule having the same biological activity when used in vivo;
a corresponding molecule, wherein said loss of immunogenicity is achieved by removing one or more T-cell epitopes, preferably one T-cell epitope, derived from the originally non-modified molecule and/or by reduction in numbers of MHC allotypes able to bind peptides derived from said molecule;
a corresponding molecule, wherein said originally present T-cell epitopes are MHC class II ligands or peptide sequences which show the ability to stimulate or bind T-cells via presentation on MHC class II;
a corresponding molecule, wherein said ligands or peptide sequences are 13 mer or 15 mer peptides;
a correspondingly molecule, wherein said peptide sequences are selected from the group as depicted in FIG. 1.
a corresponding molecule, wherein 1-9 amino acid residues, preferably one amino acid residue, in any of the originally present T-cell epitopes are altered;
a corresponding molecule, wherein the alteration of the amino acid residues is substitution, addition or deletion, preferably substitution, of originally present amino acid(s) residue(s) by other amino acid residue(s) at specific position(s);
a corresponding molecule, wherein one or more of the amino acid residue substitutions are made as indicated in FIG. 2, and, in addition, optionally one or more of the amino acid residue substitutions are carried out as indicated in FIG. 3 for the reduction in the number of MHC allotypes able to bind peptides derived from said molecule;
a corresponding molecule, wherein additionally further alteration, such as substitution, addition or deletion is conducted to restore biological activity of said molecule;
a corresponding modified molecule, wherein the amino acid alteration is made with reference to an homologous protein sequence or with reference to in silico modeling techniques;

a modified molecule having the biological activity of human interferon alpha 2 (INFα2) and being substantially non-immunogenic or less immunogenic than any non-modified molecule having the same biological activity when used in vivo, obtainable by alteration of one or more amino acids in the primary sequence by (i) removing one or more T-cell epitopes derived from the originally non-modified molecule and being MHC class II ligands or peptide sequences which show the ability to stimulate or bind T-cells via presentation on MHC class II, and/or (ii) by reduction in numbers of MHC allotypes able to bind peptides derived from said molecule, wherein said modified molecule comprises alterations which are made at one or more positions within-. following strings of contiguous amino acid residues of said primary sequence derived from the INF□2 wild-type:

(a) ISLFSCLKDRHDFGFPQEEFGNQFQKAETIPVLH (R1),
(b) FNLFSTKDSSAAWDE (R2),
(c) KEDSILAVRKYFQRITLY (R3);

a corresponding molecule, wherein said alteration is substitution of 1-9 amino acid residues;
a corresponding molecule, wherein said substitution is conducted at one or more amino acid residues from the strings R1, R2 and R3, preferably R2 and R3, and more preferably R3;
a corresponding molecule, wherein additionally one or more substitutions of amino acid residues outside the sequence strings R1, R2 or R3 are conducted;
a corresponding molecule comprising an amino acid residue substitution made at one or more positions in the wild-type molecule: 24, 26, 27, 38, 55, 63, 64, 66, 67, 76, 84, 85, 89, 103, 110, 111, 116, 117, 119, 122, 123, 126, 128, 129, 130, 153, preferably at one or more of the following positions 26, 27, 38, 63, 85, 89, 103, 110, 111, 116, 117, 122, 123, 126, 128, 153, more preferably 103, 110, 111, 116, 117, 122, 123, 126, 128, 153;
a preferred embodiment, wherein said substitution is made at one or more positions selected from 26, 27, 38 and additionally at one or more positions selected from 103, 110, 111, 116, 117, 122, 123, 126, 128, 153, or alternatively, selected from 63, 85, 89 and 103, 110, 111, 116, 117, 122, 123, 126, 128, 153;
a corresponding molecule, wherein said substitution is made at one or more positions as specified in FIG. 4;
a corresponding molecule, wherein said substitution is made at positions L26P, F27S, F38E and/or I63T, Y85S, Y89D, Y89E, Y89N and/or V103E, L110G, M111T, M111S, M111E, I116S, I116Q, L117G, L117A, Y122E, Y122Q, F123H, I126A, L128A, L153S;
a preferred corresponding molecule wherein said substitution is made at positions L26P, F27S, F38E and/or I63T, Y85S, Y89D, Y89E, Y89N and/or V103E, L110G, M111T, M111S, M111E, I116S, I116Q, L117G, L117A;
a modified molecule having the biological activity of human interferon alpha 2 (INFα2) and being substantially non-immunogenic or less immunogenic than any non-modified molecule having the same biological activity when used in vivo, obtainable by substitution of one or more amino acids in the primary sequence by (i) removing one or more T-cell epitopes derived from the originally non-modified molecule and being MHC class II ligands or peptide sequences which show the ability to stimulate or bind T-cells via presentation on MHC class II, and/or (ii) by reduction in numbers of MHC allotypes able to bind peptides derived from said molecule, wherein said substitution is made at one or more positions in a wild-type molecule INFα2a or INFα2b corresponding to at least one of the groups selected from:

(i) I24P, L26P, F27S, F38E, V55A,

(ii) I63T, L66A, F67D, F67E, W76H, F84D, F84E, Y85S, Y89D, Y89E, Y89N,

(iii) any position within sequence R3;
a corresponding molecule, whereby one or more of the following substitutions are made within sequence R3: V103E, L110G, L110S, M111T, M111S, M111E, I116S, I116Q, L117G, L117A, V119A, Y122Q, Y122E, Y122H, F123H, I126A, L128A, Y129N, L130G, L130, L153S, preferably Y122E, Y122Q, F123H, I126A, L128A, optionally containing additional amino acid residue alterations, preferably substitutions, which lead to a further diminished immunogenicity;

a modified human interferon alpha 2 (INFα2) having reduced immunogenicity consisting of the following sequence:

CDLPQTHSLGSRRTLMLLAQMRX⁰ISLFSCLKDRHDFGFPQEEFGNQFQK
AETIPVLHEMIQQIFNLFSTKDSSAAWDETLLDKFYTELYQQLNDLEACV
IQGVGVTETPLMKEDSILAVRKX¹ X²QRX³TX⁴YLKEKKYSPCAWEVVRA
EIMRSFSLSTNLQESLRSKE,

wherein
X⁰ is R, K;
X¹ is Y, E, Q;
X² is F, H;
X³ is I, A; and
X⁴is L, A;
   whereby simultaneously X¹=Y, X²=F, X³=I and X⁴=L are excluded (this sequence corresponds to the wild-type IFNα2);

a modified human interferon alpha 2 (INFα2) having reduced immunogenicity consisting of the following sequence:

CDLPQTHSLGSRRTLMLLAQMRX⁰ISLFSCLKDRHDFGFPQEEFGNQFQK
AETIPVLHEMIQQIFNJLFSTKDSSAAWDETLLDKFYTELYQQLNDLEAC
VIQGVGVTETPX¹X²KEDSX³X⁴AVRKX⁵X⁶QRX⁷TX⁸YLKEKKYSPCAW
EVVRAEIMRSFSX9STNLQESLRSKE,

wherein
X⁰ is R, K;
X¹ is L, S, G,
X² is M, T, S, E,
X³ is I, S, Q,
X⁴ is L, G,
X⁵ is Y, E, Q;
X⁶ is F, H;
X⁷ is I, A;
X⁸ is L, A; and,
X⁹ is L, S
   whereby simultaneously X¹=L, X²=M, X³=I, X⁴=L, X⁵=Y, X⁶=F, X⁷=I, X⁸=L and X⁹=L are excluded;

a modified human interferon alpha 2 (INFα2) having reduced immunogenicity consisting of the following sequence:

CDLPQTHSLGSRRTLMLLAQMRX⁰ISLFSCLKDFGFPQEEFGNQFQKAET
IPVLHEMIQQX¹X²NX³X⁴STKDSSAAX⁵DETLLDKX⁶X⁷TELX⁸QQLND
LEACVIQGVGVTETPLMKEDSILAVRKYFQRITLYLKEKKYSPCAWEVVR
AEIMRSFSLSTNLQESLRSKE,

wherein
X⁰ is R, K;
X¹ is I, T;
X² is F, D, A;
X³ is L, A;
X⁴ is F, D, E;
X⁵ is W, H;
X⁶ is F, D, E;
X⁷ is Y, S and
X⁸ is Y, D, E, N;
   whereby simultaneously X¹=I, X²=F, X³=L, X⁴=F, X⁵=W, X⁶=F, X⁷=Y and X⁸=Y are excluded;

a modified human interferon alpha 2 (INFα2) having reduced immunogenicity consisting of the following sequence:

CDLPQTHSLGSRRTLMLLAQMRX⁰ISLFSCLKDRHDFGFPQEEFGNQFQK
AETIPVLHEMIQQX¹FNLFSTKDSSAAWDETLLDKFX²TELX³QQLNDLE
ACVIQGVGVTETPLMKEDSILAVRKYFQRITLYLKEKKYSPCAWEVVRAE
IMRSFSLSTNLQESLRSKE,

wherein
X⁰ is R, K;
X¹ is I, T;
X² is Y, S and
X³ is Y, D, E, N;
   whereby simultaneously X¹=I, X²=Y and X³=Y are excluded;

a modified human interferon alpha 2 (INFα2) having reduced immunogenicity consisting of the following sequence:

CDLPQTHSLGSRRTLMLLAQMRX⁰ISX¹X²SCLKDRHDFGX³PQEEFGNQ
FQKAETIPX⁴LHEMIQQIFNLFSTKDSSAAWDETLLDKFYTELYQQLNDL
EACVIQGVGVTETPLMKEDSILAVRKYFQRITLYLKEKKYSPCAWEVVRA
EIMRSFSLSTNLQESLRSKE,

wherein
X⁰ is R, K;
X¹ is; L, P,
X² is; F, S,
X³ is F, E and
X⁴ is; V, A
   whereby simultaneously X¹=L, X²=F, X³=F and X⁴=V are excluded; it should be pointed out that all exclusion specified in the above formulas refer to and shall include all non-deimmunized versions of IFNα2 which are known in the prior art;
corresponding modified INFα2 sequences, wherein additional substitutions are made especially by the combinations as indicated in the claims;
a corresponding modified INFα2 sequence, wherein additional substitutions are made, preferably at one ore more positions within partial sequence R1 and/or R2 and /or R3 (whereby R1, R2, R3 are defined as indicated above);
a DNA sequence coding for a modified INFα2 as described above and below;
a pharmaceutical composition comprising a modified molecule having the biological activity of INFα2 as defined above, optionally together with a pharmaceutically acceptable carrier, diluent or excipient;
a method for manufacturing a modified molecule having the biological activity of INFα2 as defined above and below comprising the following steps: (i) determining the amino acid sequence of the polypeptide or part thereof,

(ii) identifying one or more potential T-cell epitopes within the amino acid sequence of the protein by any method including determination of the binding of the peptides to MHC molecules using in vitro or in silico techniques or biological assays, (iii) designing new sequence variants with one or more amino acids within the identified potential T-cell epitopes modified in such a way to substantially reduce or eliminate the activity of the T-cell epitope as determined by the binding of the peptides to MHC molecules using in vitro or in silico techniques or biological assays ,or by binding of peptide-MHC complexes to T-cells, (iv) constructing such sequence variants by recombinant DNA techniques and testing said variants in order to identify one or more variants with desirable properties, and (v) optionally repeating steps (ii)-(iv);
a corresponding method, wherein step (iii) is carried out by substitution, addition or deletion of 1-9 amino acid residues in any of the originally present T-cell epitopes or with reference to an homologous protein sequence and/or in silico modeling techniques;
a corresponding method, wherein step (ii) is carried out by the following steps: (a) selecting a region of the peptide having a known amino acid residue sequence; (b) sequentially sampling overlapping amino acid residue segments of predetermined uniform size and constituted by at least three amino acid residues from the selected region; (c) calculating MHC Class II molecule binding score for each said sampled segment by summing assigned values for each hydrophobic amino acid residue side chain present in said sampled amino acid residue segment; and (d) identifying at least one of said segments suitable for modification, based on the calculated MHC Class II molecule binding score for that segment, to change overall MHC Class II binding score for the peptide without substantially the reducing therapeutic utility of the peptide;
a corresponding method, wherein step (c) is carried out by using a Böhm scoring function modified to include 12-6 van der Waal's ligand-protein energy repulsive term and ligand conformational energy term by (1) providing a first data base of MHC Class II molecule models; (2) providing a second data base of allowed peptide backbones for said MHC Class II molecule models; (3) selecting a model from said first data base; (4) selecting an allowed peptide backbone from said second data base; (5) identifying amino acid residue side chains present in each sampled segment; (6) determining the binding affinity value for all side chains present in each sampled segment; and repeating steps (1) through (5) for each said model and each said backbone;
a peptide molecule, which is a T-cell epitope, consisting of 13 consecutive amino acid residues having a potential MHC class II binding activity and created from the primary sequence of non-modified INFα2, selected from the group as depicted in FIG. 1, FIG. 6a-c;
a peptide molecule consisting of 15, preferably at least 9, consecutive amino acid residues having a potential MHC class II binding activity and created from the primary sequence of non-modified INFa2, selected from any of the groups of partial sequences R1, R2, R3 or selected from FIG. 7;
a peptide molecule consisting of 9 -15 consecutive amino acid residues, having a potential MHC class II binding activity and created from the primary sequence of non-modified INFα2, whereby said molecule has a stimulation index of at least 1.8, preferably 1.8-2, more preferably>2, in a biological assay of cellular proliferation, wherein said index is taken as the value of cellular proliferation scored following stimulation by a peptide and divided by the value of cellular proliferation scored in control cells not in receipt peptide and wherein cellular proliferation is measured by any suitable means according to standard methods as described in more detail in the Examples;
a corresponding peptide molecule having such stimulation index value consisting of any of the sequence as indicated in FIG. 6 or 7;
a corresponding peptide molecule having such stimulation index value comprising at least 9 consecutive amino acid residues from any of the INFα2 partial sequences R1, R2, R3 as defined in claim above;
a use of a corresponding peptide, for the manufacture of INFα2 having substantially no or less immunogenicity than any non-modified molecule with the same or acceptably reduced degree of biological activity when used in vivo;
a pharmaceutical composition consisting of a synthetic peptide sequence as specified above and below and in the Figures having the biological activity of IFNα2, optionally together with a pharmaceutically acceptable carrier, diluent or excipient.

The term “T-cell epitope” means according to the understanding of this invention an amino acid sequence which is able to bind MHC class II, able to stimulate T-cells and/or also to bind (without necessarily measurably activating) T-cells in complex with MHC class II.

The term “peptide” as used herein and in the appended claims, is a compound that includes two or more amino acids. The amino acids are linked together by a peptide bond (defined herein below). There are 20 different naturally occurring amino acids involved in the biological production of peptides, and any number of them may be linked in any order to form a peptide chain or ring. The naturally occurring amino acids employed in the biological production of peptides all have the L-configuration. Synthetic peptides can be prepared employing conventional synthetic methods, utilizing L-amino acids, D-amino acids, or various combinations of amino acids of the two different configurations. Some peptides contain only a few amino acid units. Short peptides, e.g., having less than ten amino acid units, are sometimes referred to as “oligopeptides”. Other peptides contain a large number of amino acid residues, e.g. up to 100 or more, and are referred to as “polypeptides”. By convention, a “polypeptide” may be considered as any peptide chain containing three or more amino acids, whereas a “oligopeptide” is usually considered as a particular type of “short” polypeptide. Thus, as used herein, it is understood that any reference to a “polypeptide” also includes an oligopeptide. Further, any reference to a “peptide” includes polypeptides, oligopeptides, and proteins. Each different arrangement of amino acids forms different polypeptides or proteins. The number of polypeptides—and hence the number of different proteins—that can be formed is practically unlimited. “Alpha carbon (Cα)” is the carbon atom of the carbon-hydrogen (CH) component that is in the peptide chain. A “side chain” is a pendant group to Cα that can comprise a simple or complex group or moiety, having physical dimensions that can vary significantly compared to the dimensions of the peptide.

The invention may be applied to any INFα2 species of molecule with substantially the same primary amino acid sequences as those disclosed herein and would include therefore INFα2 molecules derived by genetic engineering means or other processes and may contain more or less than 165 amino acid residues.

INFα2 proteins such as identified from other mammalian sources have in common many of the peptide sequences of the present disclosure and have in common many peptide sequences with substantially the same sequence as those of the disclosed listing. Such protein sequences equally therefore fall under the scope of the present invention. The invention is conceived to overcome the practical reality that soluble proteins introduced into autologous organisms can trigger an immune response resulting in development of host antibodies that bind to the soluble protein. A prominent example of this phenomenon amongst others, is the clinical use INFα2. A significant proportion of human patients treated with INFα2 make antibodies despite the fact that this protein is produced endogenously [Russo, D. et al (1996) ibid; Stein, R. et al (1988) ibid]. The present invention seeks to address this by providing INFα2 proteins with altered propensity to elicit an immune response on administration to the human host. According to the methods described herein, the inventors have discovered and now disclose the regions of the INFa2 molecule comprising the critical T-cell epitopes driving the immune responses to this autologous protein.

The general method of the present invention leading to the modified INFα2 comprises the following steps:

(a) determining the amino acid sequence of the polypeptide or part thereof;

(b) identifying one or more potential T-cell epitopes within the amino acid sequence of the protein by any method including determination of the binding of the peptides to MHC molecules using in vitro or in silico techniques or biological assays;

(c) designing new sequence variants with one or more amino acids within the identified potential T-cell epitopes modified in such a way to substantially reduce or eliminate the activity of the T-cell epitope as determined by the binding of the peptides to MHC molecules using in vitro or in silico techniques or biological assays. Such sequence variants are created in such a way to avoid creation of new potential T-cell epitopes by the sequence variations unless such new potential T-cell epitopes are, in turn, modified in such a way to substantially reduce or eliminate the activity of the T-cell epitope; and

(d) constructing such sequence variants by recombinant DNA techniques and testing said variants in order to identify one or more variants with desirable properties according to well known recombinant techniques.

The identification of potential T-cell epitopes according to step (b) can be carried out according to methods describes previously in the prior art. Suitable methods are disclosed in WO 98/59244; WO 98/52976; WO 00/34317 and may preferably be used to identify binding propensity of INFα2a -derived peptides to an MHC class II molecule.

Another very efficacious method for identifying T-cell epitopes by calculation is described in the EXAMPLE 1 which is a preferred embodiment according to this invention.

In practice a number of variant INFα2 proteins will be produced and tested for the desired immune and functional characteristic. The variant proteins will most preferably be produced by recombinant DNA techniques although other procedures including chemical synthesis of INFα2 fragments may be contemplated.

The results of an analysis according to step (b) of the above scheme and pertaining to the human INFα2a protein sequence of 165 amino acid residues is presented in FIG. 1. The results of a design and constructs according to step (c) and (d) of the above scheme and pertaining to the modified molecule of this invention is presented in FIGS. 2 and 3.

The invention relates to INFα2 analogues in which substitutions of at least one amino acid residue have been made at positions resulting in a substantial reduction in activity of or elimination of one or more potential T-cell epitopes from the protein. One or more amino acid substitutions at particular points within any of the potential MHC class II ligands identified in Table 1 may result in a INFα2 molecule with a reduced immunogenic potential when administered as a therapeutic to the human host.

It is most preferred to provide an INFα2 molecule in which amino acid modification (e.g. a substitution) is conducted within the most immunogenic regions of the parent molecule. The inventors herein have discovered that the most immunogenic regions of the INFα2 molecule in man are confined to three regions R1, R2 and R3 comprising respectively amino acid sequences; ISLFSCLKDRHDFGFPQEEFGNQFQKAETIPVLH; FNLFSTKDSSAAWDE and KEDSILAVRKYFQRITLY. The major preferred embodiments of the present invention comprise INFa2 molecules for which the MHC class II ligands of FIG. 1 and which align either in their entirety or to a minimum of 9 amino acid residues with any of the above sequence elements R1, R2 or R3 are altered such as to eliminate binding or otherwise reduce the numbers of MHC allotypes to which the peptide can bind.

The preferred embodiments of the invention include the specific substitutions of FIG. 4. It is particularly preferred to provide modified INFα2 molecules containing combinations of substitutions from FIG. 4. Combinations which comprise modification to each of the immunogenic regions R1, R2 and R3 are preferred, and combinations comprising modifications to R2 and R3 are especially preferred although such preference is not intended to limit the combinations of substitution which are considered desirable.

For the elimination of T-cell epitopes, amino acid substitutions are preferably made at appropriate points within the peptide sequence predicted to achieve substantial reduction or elimination of the activity of the T-cell epitope. In practice an appropriate point will preferably equate to an amino acid residue binding within one of the pockets provided within the MHC class II binding groove.

It is most preferred to alter binding within the first pocket of the cleft at the so-called P1 or P1 anchor position of the peptide. The quality of binding interaction between the P1 anchor residue of the peptide and the first pocket of the MHC class II binding groove is recognized as being a major determinant of overall binding affinity for the whole peptide. An appropriate substitution at this position of the peptide will be for a residue less readily accommodated within the pocket, for example, substitution to a more hydrophilic residue. Amino acid residues in the peptide at positions equating to binding within other pocket regions within the MHC binding cleft are also considered and fall under the scope of the present.

It is understood that single amino acid substitutions within a given potential T-cell epitope are the most preferred route by which the epitope may be eliminated. Combinations of substitution within a single epitope may be contemplated and for example can be particularly appropriate where individually defined epitopes are in overlap with each other. Moreover, amino acid substitutions either singly within a given epitope or in combination within a single epitope may be made at positions not equating to the “pocket residues” with respect to the MHC class II binding groove, but at any point within the peptide sequence. Substitutions may be made with reference to an homologues structure or structural method produced using in silico techniques known in the art and may be based on known structural features of the molecule according to this invention. All such substitutions fall within the scope of the present invention.

Amino acid substitutions other than within the peptides identified above may be contemplated particularly when made in combination with substitution(s) made within a listed peptide. For example a change may be contemplated to restore structure or biological activity of the variant molecule. Such compensatory changes and changes to include deletion or addition of particular amino acid residues from the INFα2 polypeptide resulting in a variant with desired activity and in combination with changes in any of the disclosed peptides fall under the scope of the present.

In as far as this invention relates to modified INFα2, compositions containing such modified INFα2 proteins or fragments of modified INFα2 proteins and related compositions should be considered within the scope of the invention. In another aspect, the present invention relates to nucleic acids encoding modified INFα2 entities. In a further aspect the present invention relates to methods for therapeutic treatment of humans using the modified INFα2 proteins.

### SHORT DESCRIPTION OF THE FIGURES

The invention will now be illustrated, but not limited, by the following examples. The examples refer to the following drawings. The amino acid residues are consequently depicted as one-letter code.

FIG. 1 provides peptide sequences in human INFα2a with potential human MHC class II binding activity.

FIG. 2 provides substitutions leading to the elimination of T-cell epitopes of human INFα2a (WT=wild-type residue).

FIG. 3 provides additional substitutions leading to the removal of a potential T-cell epitope for one or more MHC allotypes.

FIG. 4 provides preferred substitutions in human INFα2a (WT=wild-type residue, MUT=desired residue).

FIG. 5 provides a table of the INFα2 13-mer synthetic peptides sequences analysed using an MHC class II in vitro binding assay of EXAMPLE 2.

FIG. 6 shows the results of in vitro MHC peptide binding assays for MHC allotypes. a) indicates peptides with high affinity binding (0% inhibition by competitor reference peptide) for each of the MHC allotypes tested; b) indicates peptides with medium affinity (0-50% inhibition by competitor) binding for each of the MHC allotypes tested; c) indicates peptides with low (50-100% inhibition by competitor) affinity binding for each of the MHC allotypes tested and d) indicates peptides with no detectable binding to the MHC allotypes tested.

FIG. 7 provides a table of the INFα2 15-mer peptide sequences analysed using the naïve human in vitro T-cell assay of EXAMPLE 3. The peptide ID# and position of the N-terminal peptide residue within the INFα2 sequence is indictated.

FIG. 8 shows cumulative stimulation indexes from 6 individuals that respond to stimulation with IFNα peptides. Six donors from 20 screened responded to stimulation with one or more of 51 15 mer peptides from the IFNα sequence. Responses to individual peptides are grouped into three distinct regions with region three containing the most immunogenic peptides #38 and #39 (arrows). Control peptides C32 (DRB1-restricted) and C49 (DP-restricted) are included for comparison. Cross-hatching within each bar indicates the contribution from individual donors.

FIG. 9 shows the immunogenic regions within INFα and details the peptide sequences from these regions able to stimulate naïve human T-cells.

FIG. 10 provides a table indicating INFα peptides capable of promoting proliferation of naïve human T-cells in vitro. For 5 of the donors, responses are recorded to multiple overlapping peptides from the major epitope regions R1, R2 and R3. For 3 of the donors, responses are recorded to individual synthetic peptides from R1, R2 or R3.

FIG. 11 provides a table showing frequency of MHC class II alleles in the responding and non-responding donors to IFNα peptides. a=Numerator is number of donors with DR allele, denominator is number of donors that showed T cell proliferation in vitro to that peptide (total number of responding donors=6). b=Frequency of allele in donor population. Peptides for which two or fewer responses were recorded were not evaluated. All responding donors tested negative for DRB1*14. The DRB1*14 allotype has a frequency of 1.5% in the 20 donors tested. Allorestriction of a given peptide is determined by the frequency of an allele in the donor population and the number of responding donors that express the same allele. If a peptide is associated with any particular allele (allorestricted) then the percentage shown would be expected to be greater than the frequency for the allele in the population.

FIG. 12 provides tables of IC₅₀ values for 15-mer synthetic peptides in competition binding assay for particular MHC class II allotypes.

(a) Competition MHC class II peptide binding assay to determine relative binding affinities of INFα peptides capable of promoting proliferation of naïve human T-cells in vitro to DRB1*0101. INFα peptides were incubated with fixed HOM-2 cells in the presence of 10 μM biotinylated influenza haemagglutinin 307-319. The concentration of competitor peptide causing 50% inhibition of maximun biotinylated peptide binding was taken as the IC₅₀. Influenza 103-115 was included as a high affinity control. IC₅₀≦20 μM=high affinity, IC₅₀=20-100 μM=Medium Affinity, IC₅₀≧100 uM=Low Affinity.

(b) Competition MHC class II peptide binding assay to determine relative binding affinities of INFα peptides capable of promoting proliferation of naïve human T-cells in vitro to DRB1*0701. INFα peptides were incubated with fixed MOU (MANN) cells in the presence of 10 uM biotinylated tetanus toxin 828-840. The concentration of competitor peptide causing 50% inhibition of maximun biotinylated peptide binding was taken as the IC₅₀. Tetanus toxin 828-840 was included as a high affinity control. IC₅₀≦20 μM=high affinity, IC₅₀=20-100 μM=Medium Affinity, IC₅₀≧100 μM=Low Affinity.

(c) Competition MHC class II peptide binding assay to determine relative binding affinities of INFα peptides capable of promoting proliferation of naïve human T-cells in vitro to DRB1*0401. INFα peptides were incubated with fixed WT-51 cells in the presence of 50 uM biotinylated influenza haemagglutinin 307-319. The concentration of competitor peptide causing 50% inhibition of maximun biotinylated peptide binding was taken as the IC₅₀. Influenza 103-115 was included as a high affinity control. IC₅₀≦20 μM=high affinity, IC₅₀=20-100 μM=Medium Affinity, IC₅₀≧100 μM=Low Affinity.

FIG. 13 provides a table detailing substitutions within INFα which provide molecules with retained activity in the anti-proliferation assay of EXAMPLE 7. WT=wild-type residue; #=residue number; Mut=mutation conducted. Epitope Region indicates location of substitution with respect to immunogenic epitope regions R1, R2 or R3.

FIG. 14 provides representative data of the anti-proliferative effect of selected mutant INFα2 molecules. Assays were conducted according to the methods of EXAMPLE 7. Panel a) shows activity of molecules with substitution within immunogenic epitope R1. Panel b) shows activity of molecules with substitution within immunogenic epitope R2. Panel c) shows activity of molecules with substitution within immunogenic epitope R3.

FIG. 15 provides panels that show individual donor responses to INFα synthetic peptides. Data from control peptides C32 (DRB1-restricted) and C49 (DP-restricted) are included for comparison. Immunogenic regions R1, R2, R3 are indicated on relevant panels. Threshold for positive stimulation index=2.

In the following Examples the invention is described in more detail which shall not be interpreted as a limitation or restriction.

### EXAMPLE 1

There are a number of factors that play important roles in determining the total structure of a protein or polypeptide. First, the peptide bond, i.e., that bond which joins the amino acids in the chain together, is a covalent bond. This bond is planar in structure, essentially a substituted amide. An “amide” is any of a group of organic compounds containing the grouping —CONH—.

The planar peptide bond linking Cα of adjacent amino acids may be represented as depicted below:

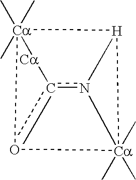

Because the O═C and the C—N atoms lie in a relatively rigid plane, free rotation does not occur about these axes. Hence, a plane schematically depicted by the interrupted line is sometimes referred to as an “amide” or “peptide plane” plane wherein lie the oxygen (O), carbon (C), nitrogen (N), and hydrogen (H) atoms of the peptide backbone. At opposite corners of this amide plane are located the Cα atoms. Since there is substantially no rotation about the O═C and C—N atoms in the peptide or amide plane, a polypeptide chain thus comprises a series of planar peptide linkages joining the Cα atoms.

A second factor that plays an important role in defining the total structure or conformation of a polypeptide or protein is the angle of rotation of each amide plane about the common Cα linkage. The terms “angle of rotation” and “torsion angle” are hereinafter regarded as equivalent terms. Assuming that the O, C, N, and H atoms remain in the amide plane (which is usually a valid assumption, although there may be some slight deviations from planarity of these atoms for some conformations), these angles of rotation define the N and R polypeptide's backbone conformation, i.e., the structure as it exists between adjacent residues. These two angles are known as φ and ψ. A set of the angles φ₁, ψ₁, where the subscript represents a particular residue of a polypeptide chain, thus effectively defines the polypeptide secondary structure. The conventions used in defining the φ, ψ angles, i.e., the reference points at which the amide planes form a zero degree angle, and the definition of which angle is φ, and which angle is ψ, for a given polypeptide, are defined in the literature (see, e.g., Ramachandran et al. Adv. Prot. Chem. 23:283-437 (1968), at pages 285-94, which pages are incorporated herein by reference).

The present method can be applied to any protein, and is based in part upon the discovery that in humans the primary Pocket 1 anchor position of MHC Class II molecule binding grooves has a well designed specificity for particular amino acid side chains. The specificity of this pocket is determined by the identity of the amino acid at position 86 of the beta chain of the MHC Class II molecule. This site is located at the bottom of Pocket 1 and determines the size of the side chain that can be accommodated by this pocket. Marshall, K. W., J. Immunol., 152:4946-4956 (1994). If this residue is a glycine, then all hydrophobic aliphatic and aromatic amino acids (hydrophobic aliphatics being: valine, leucine, isoleucine, methionine and aromatics being: phenylalanine, tyrosine and tryptophan) can be accommodated in the pocket, a preference being for the aromatic side chains. If this pocket residue is a valine, then the side chain of this amino acid protrudes into the pocket and restricts the size of peptide side chains that can be accommodated such that only hydrophobic aliphatic side chains can be accommodated. Therefore, in an amino acid residue sequence, wherever an amino acid with a hydrophobic aliphatic or aromatic side chain is found, there is the potential for a MHC Class II restricted T-cell epitope to be present. If the side-chain is hydrophobic aliphatic, however, it is approximately twice as likely to be associated with a T-cell epitope than an aromatic side chain (assuming an approximately even distribution of Pocket 1 types throughout the global population).

A computational method embodying the present invention profiles the likelihood of peptide regions to contain T-cell epitopes as follows:

(1) The primary sequence of a peptide segment of predetermined length is scanned, and all hydrophobic aliphatic and aromatic side chains present are identified. (2) The hydrophobic aliphatic side chains are assigned a value greater than that for the aromatic side chains; preferably about twice the value assigned to the aromatic side chains, e.g., a value of 2 for a hydrophobic aliphatic side chain and a value of 1 for an aromatic side chain. (3) The values determined to be present are summed for each overlapping amino acid residue segment (window) of predetermined uniform length within the peptide, and the total value for a particular segment (window) is assigned to a single amino acid residue at an intermediate position of the segment (window), preferably to a residue at about the midpoint of the sampled segment (window). This procedure is repeated for each sampled overlapping amino acid residue segment (window). Thus, each amino acid residue of the peptide is assigned a value that relates to the likelihood of a T-cell epitope being present in that particular segment (window). (4) The values calculated and assigned as described in Step 3, above, can be plotted against the amino acid coordinates of the entire amino acid residue sequence being assessed. (5) All portions of the sequence which have a score of a predetermined value, e.g., a value of 1, are deemed likely to contain a T-cell epitope and can be modified, if desired.

This particular aspect of the present invention provides a general method by which the regions of peptides likely to contain T-cell epitopes can be described. Modifications to the peptide in these regions have the potential to modify the MHC Class II binding characteristics.

According to another aspect of the present invention, T-cell epitopes can be predicted with greater accuracy by the use of a more sophisticated computational method which takes into account the interactions of peptides with models of MHC Class II alleles. The computational prediction of T-cell epitopes present within a peptide according to this particular aspect contemplates the construction of models of at least 42 MHC Class II alleles based upon the structures of all known MHC Class II molecules and a method for the use of these models in the computational identification of T-cell epitopes, the construction of libraries of peptide backbones for each model in order to allow for the known variability in relative peptide backbone alpha carbon (Cα) positions, the construction of libraries of amino-acid side chain conformations for each backbone dock with each model for each of the 20 amino-acid alternatives at positions critical for the interaction between peptide and MHC Class II molecule, and the use of these libraries of backbones and side-chain conformations in conjunction with a scoring function to select the optimum backbone and side-chain conformation for a particular peptide docked with a particular MHC Class II molecule and the derivation of a binding score from this interaction.

Models of MHC Class II molecules can be derived via homology modeling from a number of similar structures found in the Brookhaven Protein Data Bank (“PDB”). These may be made by the use of semi-automatic homology modeling software (Modeller, Sali A. & Blundell T L., 1993. J. Mol Biol 234:779-815) which incorporates a simulated annealing function, in conjunction with the CHARMm force-field for energy minimisation (available from Molecular Simulations Inc., San Diego, Calif.). Alternative modeling methods can be utilized as well.

The present method differs significantly from other computational methods which use libraries of experimentally derived binding data of each amino-acid alternative at each position in the binding groove for a small set of MHC Class II molecules (Marshall, K. W., et al., Biomed. Pept. Proteins Nucleic Acids, 1(3):157-162) (1995) or yet other computational methods which use similar experimental binding data in order to define the binding characteristics of particular types of binding pockets within the groove, again using a relatively small subset of MHC Class II molecules, and then ‘mixing and matching’ pocket types from this pocket library to artificially create further ‘virtual’ MHC Class II molecules (Sturniolo T., et al., Nat. Biotech, 17(6): 555-561 (1999). Both prior methods suffer the major disadvantage that, due to the complexity of the assays and the need to synthesize large numbers of peptide variants, only a small number of MHC Class II molecules can be experimentally scanned. Therefore the first prior method can only make predictions for a small number of MHC Class II molecules. The second prior method also makes the assumption that a pocket lined with similar amino-acids in one molecule will have the same binding characteristics when in the context of a different Class II allele and suffers further disadvantages in that only those MHC Class II molecules can be ‘virtually’ created which contain pockets contained within the pocket library. Using the modeling approach described herein, the structure of any number and type of MHC Class II molecules can be deduced, therefore alleles can be specifically selected to be representative of the global population. In addition, the number of MHC Class II molecules scanned can be increased by making further models further than having to generate additional data via complex experimentation.

The use of a backbone library allows for variation in the positions of the Cα atoms of the various peptides being scanned when docked with particular MHC Class II molecules. This is again in contrast to the alternative prior computational methods described above which rely on the use of simplified peptide backbones for scanning amino-acid binding in particular pockets. These simplified backbones are not likely to be representative of backbone conformations found in ‘real’ peptides leading to inaccuracies in prediction of peptide binding. The present backbone library is created by superposing the backbones of all peptides bound to MHC Class II molecules found within the Protein Data Bank and noting the root mean square (RMS) deviation between the Cα atoms of each of the eleven amino-acids located within the binding groove. While this library can be derived from a small number of suitable available mouse and human structures (currently 13), in order to allow for the possibility of even greater variability, the RMS figure for each C″-α position is increased by 50%. The average Cα position of each amino-acid is then determined and a sphere drawn around this point whose radius equals the RMS deviation at that position plus 50%. This sphere represents all allowed Cα positions.

Working from the Cα with the least RMS deviation (that of the amino-acid in Pocket 1 as mentioned above, equivalent to Position 2 of the 11 residues in the binding groove), the sphere is three-dimensionally gridded, and each vertex within the grid is then used as a possible location for a Cα of that amino-acid. The subsequent amide plane, corresponding to the peptide bond to the subsequent amino-acid is grafted onto each of these Cαs and the φ and ψ angles are rotated step-wise at set intervals in order to position the subsequent Cα. If the subsequent Cα falls within the ‘sphere of allowed positions’ for this Cα than the orientation of the dipeptide is accepted, whereas if it falls outside the sphere then the dipeptide is rejected.

This process is then repeated for each of the subsequent Cα positions, such that the peptide grows from the Pocket 1 Cα ‘seed’, until all nine subsequent Cαs have been positioned from all possible permutations of the preceding Cαs. The process is then repeated once more for the single Cα preceding pocket 1 to create a library of backbone Cα positions located within the binding groove.

The number of backbones generated is dependent upon several factors: The size of the ‘spheres of allowed positions’; the fineness of the gridding of the ‘primary sphere’ at the Pocket 1 position; the fineness of the step-wise rotation of the φ and ψ angles used to position subsequent Cαs. Using this process, a large library of backbones can be created. The larger the backbone library, the more likely it will be that the optimum fit will be found for a particular peptide within the binding groove of an MHC Class II molecule. Inasmuch as all backbones will not be suitable for docking with all the models of MHC Class II molecules due to clashes with amino-acids of the binding domains, for each allele a subset of the library is created comprising backbones which can be accommodated by that allele.

The use of the backbone library, in conjunction with the models of MHC Class II molecules creates an exhaustive database consisting of allowed side chain conformations for each amino-acid in each position of the binding groove for each MHC Class II molecule docked with each allowed backbone. This data set is generated using a simple steric overlap function where a MHC Class II molecule is docked with a backbone and an amino-acid side chain is grafted onto the backbone at the desired position. Each of the rotatable bonds of the side chain is rotated step-wise at set intervals and the resultant positions of the atoms dependent upon that bond noted. The interaction of the atom with atoms of side-chains of the binding groove is noted and positions are either accepted or rejected according to the following criteria: The sum total of the overlap of all atoms so far positioned must not exceed a pre-determined value. Thus the stringency of the conformational search is a function of the interval used in the step-wise rotation of the bond and the pre-determined limit for the total overlap. This latter value can be small if it is known that a particular pocket is rigid, however the stringency can be relaxed if the positions of pocket side-chains are known to be relatively flexible. Thus allowances can be made to imitate variations in flexibility within pockets of the binding groove. This conformational search is then repeated for every amino-acid at every position of each backbone when docked with each of the MHC Class II molecules to create the exhaustive database of side-chain conformations.

A suitable mathematical expression is used to estimate the energy of binding between models of MHC Class II molecules in conjunction with peptide ligand conformations which have to be empirically derived by scanning the large database of backbone/side-chain conformations described above. Thus a protein is scanned for potential T-cell epitopes by subjecting each possible peptide of length varying between 9 and 20 amino-acids (although the length is kept constant for each scan) to the following computations: An MHC Class II molecule is selected together with a peptide backbone allowed for that molecule and the side-chains corresponding to the desired peptide sequence are grafted on. Atom identity and interatomic distance data relating to a particular side-chain at a particular position on the backbone are collected for each allowed conformation of that amino-acid (obtained from the database described above). This is repeated for each side-chain along the backbone and peptide scores derived using a scoring function. The best score for that backbone is retained and the process repeated for each allowed backbone for the selected model. The scores from all allowed backbones are compared and the highest score is deemed to be the peptide score for the desired peptide in that MHC Class II model. This process is then repeated for each model with every possible peptide derived from the protein being scanned, and the scores for peptides versus models are displayed.

In the context of the present invention, each ligand presented for the binding affinity calculation is an amino-acid segment selected from a peptide or protein as discussed above. Thus, the ligand is a selected stretch of amino acids about 9 to 20 amino acids in length derived from a peptide, polypeptide or protein of known sequence. The terms “amino acids” and “residues” are hereinafter regarded as equivalent terms.

The ligand, in the form of the consecutive amino acids of the peptide to be examined grafted onto a backbone from the backbone library, is positioned in the binding cleft of an MHC Class II molecule from the MHC Class II molecule model library via the coordinates of the C″-α atoms of the peptide backbone and an allowed conformation for each side-chain is selected from the database of allowed conformations. The relevant atom identities and interatomic distances are also retrieved from this database and used to calculate the peptide binding score. Ligands with a high binding affinity for the MHC Class II binding pocket are flagged as candidates for site-directed mutagenesis. Amino-acid substitutions are made in the flagged ligand (and hence in the protein of interest) which is then retested using the scoring function in order to determine changes which reduce the binding affinity below a predetermined threshold value. These changes can then be incorporated into the protein of interest to remove T-cell epitopes.

Binding between the peptide ligand and the binding groove of MHC Class II molecules involves non-covalent interactions including, but not limited to: hydrogen bonds, electrostatic interactions, hydrophobic (lipophilic) interactions and Van der Walls interactions. These are included in the peptide scoring function as described in detail below.

It should be understood that a hydrogen bond is a non-covalent bond which can be formed between polar or charged groups and consists of a hydrogen atom shared by two other atoms. The hydrogen of the hydrogen donor has a positive charge where the hydrogen acceptor has a partial negative charge. For the purposes of peptide/protein interactions, hydrogen bond donors may be either nitrogens with hydrogen attached or hydrogens attached to oxygen or nitrogen. Hydrogen bond acceptor atoms may be oxygens not attached to hydrogen, nitrogens with no hydrogens attached and one or two connections, or sulphurs with only one connection. Certain atoms, such as oxygens attached to hydrogens or imine nitrogens (e.g. C═NH) may be both hydrogen acceptors or donors. Hydrogen bond energies range from 3 to 7 Kcal/mol and are much stronger than Van der Waal's bonds, but weaker than covalent bonds. Hydrogen bonds are also highly directional and are at their strongest when the donor atom, hydrogen atom and acceptor atom are co-linear.

Electrostatic bonds are formed between oppositely charged ion pairs and the strength of the interaction is inversely proportional to the square of the distance between the atoms according to Coulomb's law. The optimal distance between ion pairs is about 2.8 Å. In protein/peptide interactions, electrostatic bonds may be formed between arginine, histidine or lysine and aspartate or glutamate. The strength of the bond will depend upon the pKa of the ionizing group and the dielectric constant of the medium although they are approximately similar in strength to hydrogen bonds.

Lipophilic interactions are favorable hydrophobic-hydrophobic contacts that occur between he protein and peptide ligand. Usually, these will occur between hydrophobic amino acid side chains of the peptide buried within the pockets of the binding groove such that they are not exposed to solvent. Exposure of the hydrophobic residues to solvent is highly unfavorable since the surrounding solvent molecules are forced to hydrogen bond with each other forming cage-like clathrate structures. The resultant decrease in entropy is highly unfavorable. Lipophilic atoms may be sulphurs which are neither polar nor hydrogen acceptors and carbon atoms which are not polar.

Van der Waal's bonds are non-specific forces found between atoms which are 3-4 Å apart. They are weaker and less specific than hydrogen and electrostatic bonds. The distribution of electronic charge around an atom changes with time and, at any instant, the charge distribution is not symmetric. This transient asymmetry in electronic charge induces a similar asymmetry in neighboring atoms. The resultant attractive forces between atoms reaches a maximum at the Van der Waal's contact distance but diminishes very rapidly at about 1 Å to about 2 Å. Conversely, as atoms become separated by less than the contact distance, increasingly strong repulsive forces become dominant as the outer electron clouds of the atoms overlap. Although the attractive forces are relatively weak compared to electrostatic and hydrogen bonds (about 0.6 Kcal/mol), the repulsive forces in particular may be very important in determining whether a peptide ligand may bind successfully to a protein.

In one embodiment, the Böhm scoring function (SCORE1 approach) is used to estimate the binding constant. (Böhm, H. J., J. Comput Aided Mol. Des., 8(3):243-256 (1994) which is hereby incorporated in its entirety). In another embodiment, the scoring function (SCORE2 approach) is used to estimate the binding affinities as an indicator of a ligand containing a T-cell epitope (Böhm, H. J., J. Comput Aided Mol. Des., 12(4):309-323 (1998) which is hereby incorporated in its entirety). However, the Böhm scoring functions as described in the above references are used to estimate the binding affinity of a ligand to a protein where it is already known that the ligand successfully binds to the protein and the protein/ligand complex has had its structure solved, the solved structure being present in the Protein Data Bank (“PDB”). Therefore, the scoring function has been developed with the benefit of known positive binding data. In order to allow for discrimination between positive and negative binders, a repulsion term must be added to the equation. In addition, a more satisfactory estimate of binding energy is achieved by computing the lipophilic interactions in a pairwise manner rather than using the area based energy term of the above Böhm functions.

Therefore, in a preferred embodiment, the binding energy is estimated using a modified Böhm scoring function. In the modified Böhm scoring function, the binding energy between protein and ligand (ΔG_{bind}) is estimated considering the following parameters: The reduction of binding energy due to the overall loss of translational and rotational entropy of the ligand (ΔG₀); contributions from ideal hydrogen bonds (ΔG_{hb}) where at least one partner is neutral; contributions from unperturbed ionic interactions (ΔG_{ionic}); lipophilic interactions between lipophilic ligand atoms and lipophilic acceptor atoms (ΔGₗᵢₚₒ); the loss of binding energy due to the freezing of internal degrees of freedom in the ligand, i.e., the freedom of rotation about each C—C bond is reduced (ΔGᵣₒₜ); the energy of the interaction between the protein and ligand (E_{VdW}). Consideration of these terms gives equation 1:
(ΔG_{bind})=(ΔG₀)+(ΔG_{hb}×N_{hb})+(ΔG_{ionic}×N_{ionic})+(ΔGₗᵢₚₒ×Nₗᵢₚₒ)+(ΔGᵣₒₜ+Nᵣₒₜ)+(E_{VdW}).

Where N is the number of qualifying interactions for a specific term and, in one embodiment, ΔG₀, ΔG_{hb}, ΔG_{ionic}, ΔGₗᵢₚₒ and ΔGᵣₒₜ are constants which are given the values: 5.4, −4.7, −4.7, −0.17, and 1.4, respectively.

The term N_{hb} is calculated according to equation 2:
N_{hb}Σ_{h-bonds}f(ΔR, Δα)×f(N_{neighb})×f_{pcs}

f(ΔR, Δα) is a penalty function which accounts for large deviations of hydrogen bonds from ideality and is calculated according to equation 3:

$$f ( Δ R , Δ - α ) = f1 ( Δ R ) \times f2 ( Δα )$$

$$Where \text{:} \begin{matrix}f1 ( Δ R ) = 1 if Δ R <= TOL \\ or = 1 - ( Δ R - TOL ) / 0.4 if Δ R <= 0.4 + TOL \\ or = 0 if Δ R > 0.4 + TOL\end{matrix}$$

$$And \text{:} \begin{matrix}f2 ( Δα ) = 1 if Δα < 30 ° \\ or = 1 - ( Δα - 30 ) / 50 if Δα <= 80 ° \\ or = 0 if Δα > 80 °\end{matrix}$$

TOL is the tolerated deviation in hydrogen bond length=0.25 Å

ΔR is the deviation of the H—O/N hydrogen bond length from the ideal value=1.9 Å

Δα is the deviation of the hydrogen bond angle ∠_{N/O—H . . . O/N} from its idealized value of 180°

f(N_{neighb}) distinguishes between concave and convex parts of a protein surface and therefore assigns greater weight to polar interactions found in pockets rather than those found at the protein surface. This function is calculated according to equation 4 below:
f(N_{neighb})=(N_{neighb}/N_{neighb,0})^{α} where α=0.5

N_{neighb} is the number of non-hydrogen protein atoms that are closer than 5 Å to any given protein atom.

N_{neighb,0} is a constant=25

f_{pcs} is a function which allows for the polar contact surface area per hydrogen bond and therefore distinguishes between strong and weak hydrogen bonds and its value is determined according to the following criteria:
f_{pcs}=β when Aₚₒₗₐᵣ/N_{HB}<10 Å²
or
f_{pcs}=l when Aₚₒₗₐᵣ/N_{HB}>10 Å²

Aₚₒₗₐᵣ is the size of the polar protein-ligand contact surface

N_{HB} is the number of hydrogen bonds

β is a constant whose value=1.2

For the implementation of the modified Böhm scoring function, the contributions from ionic interactions, ΔG_{ionic}, are computed in a similar fashion to those from hydrogen bonds described above since the same geometry dependency is assumed.

The term Nₗᵢₚₒ is calculated according to equation 5 below:
Nₗᵢₚₒ=Σ_{lL}f(r_{lL})

f(r_{lL}) is calculated for all lipophilic ligand atoms, l, and all lipophilic protein atoms, L, according to the following criteria:
f(r_{lL})=1 when r_{lL}<=R1f(r_{lL})=(r_{lL}−R1)/(R2−R1) when R2<r_{lL}>R1
f(r_{lL})=0 when r_{lL}>=R2
Where:
R1=rₗ^{vdw}+r_{L}^{vdw}+0.5
and
R2=R1+3.0

rₗ^{vdw} is the Van der Waal's radius of atom l

and r_{L}^{vdw} is the Van der Waal's radius of atom L

The term Nᵣₒₜ is the number of rotable bonds of the amino acid side chain and is taken to be the number of acyclic sp³-sp³ and sp³-sp² bonds. Rotations of terminal —CH₃ or —NH₃ are not taken into account.

The final term, E_{VdW}, is calculated according to equation 6 below:
E_{VdW}=ε₁ε₂((r₁^{vdw}+r₂^{vdw})¹²/r¹²−(r₁^{vdw}+r₂^{vdw})⁶/r⁶), where:

ε₁ and ε₂ are constants dependant upon atom identity

r₁^{vdw}+r₂^{vdw} are the Van der Waal's atomic radii

r is the distance between a pair of atoms.

With regard to Equation 6, in one embodiment, the constants ε₁ and ε₂ are given the atom values: C: 0.245, N: 0.283, O: 0.316, S: 0.316, respectively (i.e. for atoms of Carbon, Nitrogen, Oxygen and Sulphur, respectively). With regards to equations 5 and 6, the Van der Waal's radii are given the atom values C: 1.85, N: 1.75, O: 1.60, S: 2.00 Å.

It should be understood that all predetermined values and constants given in the equations above are determined within the constraints of current understandings of protein ligand interactions with particular regard to the type of computation being undertaken herein. Therefore, it is possible that, as this scoring function is refined further, these values and constants may change hence any suitable numerical value which gives the desired results in terms of estimating the binding energy of a protein to a ligand may be used and hence fall within the scope of the present invention.

As described above, the scoring function is applied to data extracted from the database of side-chain conformations, atom identities, and interatomic distances. For the purposes of the present description, the number of MHC Class II molecules included in this database is 42 models plus four solved structures. It should be apparent from the above descriptions that the modular nature of the construction of the computational method of the present invention means that new models can simply be added and scanned with the peptide backbone library and side-chain conformational search function to create additional data sets which can be processed by the peptide scoring function as described above. This allows for the repertoire of scanned MHC Class II molecules to easily be increased, or structures and associated data to be replaced if data are available to create more accurate models of the existing alleles.

The present prediction method can be calibrated against a data set comprising a large number of peptides whose affinity for various MHC Class II molecules has previously been experimentally determined. By comparison of calculated versus experimental data, a cut of value can be determined above which it is known that all experimentally determined T-cell epitopes are correctly predicted.

It should be understood that, although the above scoring function is relatively simple compared to some sophisticated methodologies that are available, the calculations are performed extremely rapidly. It should also be understood that the objective is not to calculate the true binding energy per se for each peptide docked in the binding groove of a selected MHC Class II protein. The underlying objective is to obtain comparative binding energy data as an aid to predicting the location of T-cell epitopes based on the primary structure (i.e. amino acid sequence) of a selected protein. A relatively high binding energy or a binding energy above a selected threshold value would suggest the presence of a T-cell epitope in the ligand. The ligand may then be subjected to at least one round of amino-acid substitution and the binding energy recalculated. Due to the rapid nature of the calculations, these manipulations of the peptide sequence can be performed interactively within the program's user interface on cost-effectively available computer hardware. Major investment in computer hardware is thus not required. It would be apparent to one skilled in the art that other available software could be used for the same purposes. In particular, more sophisticated software which is capable of docking ligands into protein binding-sites may be used in conjunction with energy minimization. Examples of docking software are: DOCK (Kuntz et al., J. Mol. Biol., 161:269-288 (1982)), LUDI (Böhm, H. J., J. Comput Aided Mol. Des., 8:623-632 (1994)) and FLEXX (Rarey M., et al., ISMB, 3:300-308 (1995)). Examples of molecular modeling and manipulation software include: AMBER (Tripos) and CHARMn (Molecular Simulations Inc.). The use of these computational methods would severely limit the throughput of the method of this invention due to the lengths of processing time required to make the necessary calculations. However, it is feasible that such methods could be used as a ‘secondary screen’ to obtain more accurate calculations of binding energy for peptides which are found to be ‘positive binders’ via the method of the present invention.

The limitation of processing time for sophisticated molecular mechanic or molecular dynamic calculations is one which is defined both by the design of the software which makes these calculations and the current technology limitations of computer hardware. It may be anticipated that, in the future, with the writing of more efficient code and the continuing increases in speed of computer processors, it may become feasible to make such calculations within a more manageable time-frame.

Further information on energy functions applied to macromolecules and consideration of the various interactions that take place within a folded protein structure can be found in: Brooks, B. R., et al., J. Comput. Chem., 4:187-217 (1983) and further information concerning general protein-ligand interactions can be found in: Dauber-Osguthorpe et al., Proteins 4(1):31-47(1988), which are incorporated herein by reference in their entirety. Useful background information can also be found, for example, in Fasman, G. D., ed., Prediction of Protein Structure and the Principles of Protein Conformation, Plenum Press, New York, ISBN: 0-306 4313-9.

The following examples describe the invention in more detail.

### EXAMPLE 2

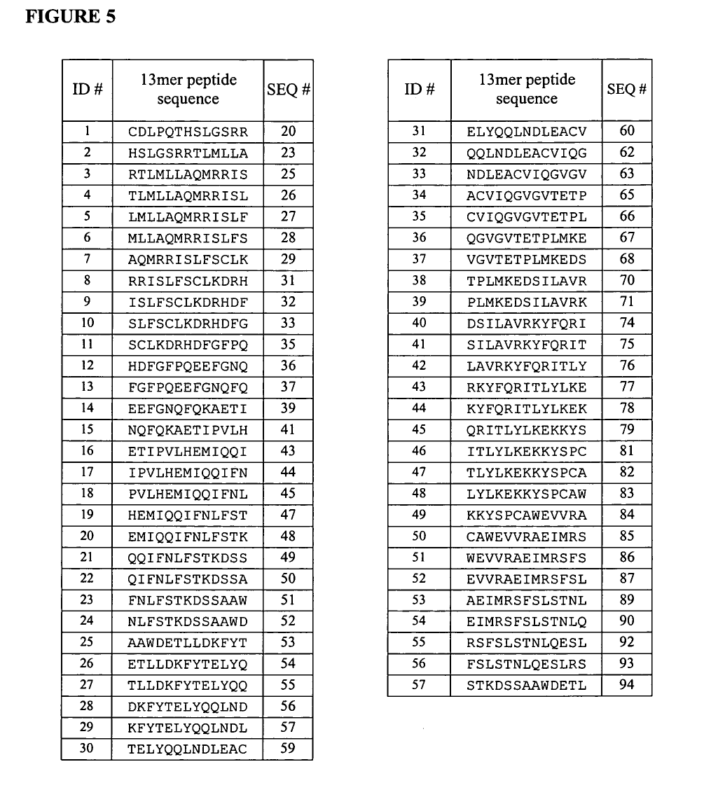

The 165 amino acid sequence of INFα2 was analyzed in silico broadly by the method of EXAMPLE 1. A panel of 57 13-mer synthetic peptides were produced and analyzed for their ability to bind in vitro with human MHC class II molecules. The peptide sequences are depicted in FIG. 5.

MHC class II synthetic peptide binding assays were conducted using human lymphoblastoid B cells of known HLA-DR allotype. Cells were fixed with paraformaldeyde and incubated with either biotinylated peptides alone or with a non-biotinylated competitor peptide to determine IC₅₀ values. Following incubation with the peptides, cells were lysed and the MHC Class II molecules captured by the anti-HLA-DR α-chain monoclonal antibody LB3.1. Bound biotinylated peptide was detected by streptavidin peroxidase, and the amount of bound peptide quantitated by a luminescent read out.

Competition-binding assays were conducted, where non-biotinylated test peptides were incubated with the fixed cells in the presence of the biotinylated competitor peptide. Competitor peptides were previously determined to have IC₅₀ values for the particular allotypes of interest using a simple (non-competitive) binding assay. The IC₅₀ value is the concentration of the unlabeled peptide that prevents 50% of the labelled peptide from binding. The concentration of the biotinylated peptide was determined experimentally to be at least one sixth of its measured ED₅₀ (concentration of peptide that gives one half of the maximum response) for each allele, to ensure that the inhibition was primarily measuring the binding characteristics of the competitor peptide.

EBV transformed human B lymphoblastoid cell lines are obtainable from ECACC (Salisbury, UK). HOM-2 cells were used in assays for DRB1*0101 binding; WT51 cells were used in assays of DRB1*0401 binding and MOU (MANN) cells were used for assays of DRB1*0701 binding. The mouse hybridoma LB3.1 was obtained from the American Tissue Culture Collection ATCC (Virginia, USA). Enhanced Chemiluminescent (ECL) reagent was purchased from Amersham Pharmacia (Amersham, UK). RPMI 1640 medium, L-glutamine, and penicillin/streptomycin were obtained from Life Technologies (Paisley, UK). Optiplates™ were obtained from Packard (Pangbourne, England). Biotinylated peptides were obtained from Babraham Tech^{nix} (Cambridge, England) and non-biotinylated peptides from Pepscan Systems (Lelystad, The Netherlands). Prosep A was obtained from Millipore (Watford, UK). DAB, PMSF, iodoacetamide, benzamidine, leupeptin, pepstatin, PBS tablets, DMSO, BSA, streptavidin peroxidase conjugate and all other chemicals were obtained from The Sigma Chemical Company (Poole, UK).

Lymphoblastoid cells were cultivated in RPMI-1640 medium plus 10% foetal bovine serum (FBS), L-glutamine, and penicillin/streptomycin in a humidified atmosphere at 37° C./5% CO₂.

LB3.1 hybridoma cells were cultivated in RPMI-1640 medium plus 10% foetal bovine serum (FBS), L-glutamine, and penicillin/streptomycin in a humidified atmosphere at 37° C./5% CO2 and LB3.1 antibody purified from the culture supernatant. The supernatant was filtered using 0.22 μM filters then 50 ml of 1M Tris pH 8.0 was added per 450 ml making a 0.1M-buffered solution. The buffered supernatant was then passed through a 3 ml PROSEP A column overnight at 4° C. and washed with 25 ml of PBS. LB3.1 antibody was eluted using 8 ml of 0.1M citrate pH 3.0, and each 0.5 ml fraction was collected into 500 μl 1M Tris-HCl pH 8.0. The protein content of each fraction was determined using a spectrophotometer (A280 nm). Fractions were pooled and dialysed into 800 ml PBS, using a Slide-A-Lyser 3.5K cut-off (Pierce). Purity of the LB3.1 was checked by reduced SDS-PAGE followed by Coomassie staining.

Binding assays for each peptide/allotype combination were conducted in triplicate in 96-well flat bottom Optiplates™ using 2×10⁶ cells per well. Cells were washed twice with RPMI-1640 then fixed with 0.5% paraformaldehyde/PBS for 30 min on ice. After 2 washes with RPMI-1640 the cells were incubated with either: biotinylated peptide; biotinylated peptide+non-biotinylated competitive peptide or no peptide. Incubation was conducted using Peptide Binding Buffer (100 mM Citrate/Phosphate pH 4.5, 5 mM EDTA, 1 mM PMSF, 100 μM Leupeptin, 1 mM Iodoacetamide, 100 μM Pepstatin A, 1 mM Benzamidine) at 37° C. for 24 h. Cells were collected by centrifugation, then 80 μl supernatant was removed and replaced with 80 μl/well of NP40 lysis buffer (0.5% NP40, 150 mM NaCl, 1 mM PMSF, 100 μM Leupeptin, 1 mM Iodoacetamide, 100 μM Pepstatin A, 1 mM Benzamidine 50 mM Tris-HCl pH to 8.0). Cells were incubated at 4° C. for 45 minutes and a cleared lysate obtained by centrifugation. 50 μl (triplicate/sample) of lysate was added to each well of a pre-coated 96 well assay plate containing 50 μl PBS/5% BSA. In all experiments pre-coating had been conducted the night before where assay plates had been pre-treated at 4° C. with 100 μl/well anti-class II antibody LB3.1 diluted to 20 μg/ml in PBS. Excess antibody was removed and the plate blocked with 250 μl/well of PBS/5% BSA for 2 hours at room temperature. Each plate was washed 7× with PBS/1% Tween and 50 μl of PBS/5% BSA/0.5% NP40 added to each well before addition of the cell lysates.

Following addition of the lysates, plates were incubated at 4° C. for 2 hours, then washed 7× with PBS/0.1% Tween. 100 μl of Streptavidin Peroxidase diluted 1:1000 in PBS/5% BSA/ 0.1% Tween was added to each well and the plates incubated at 4° C. for 1 hour. Plates were washed 7× with PBS/0.1% Tween and 100 μl of chemiluminescent substrate (Amersham Pharmacia) added to each well. Plates were read using a Perkin Elmer MicroBeta® TriLux plate reader and results were given in CPS.

In the competition analyses: maximum binding of biotinylated peptide was defined as the binding (CPS) occurring in the absence of the competitor peptide, and inhibition by the formula:

$$\% Inhibition = 100 \times \frac{[ ( CPS with no competitor ) - ( CPS with competitor ) ]}{[ CPS with no competitor ]}$$

The concentration of competitor peptide causing 50% inhibition of maximun biotinylated peptide binding was taken as the IC₅₀.

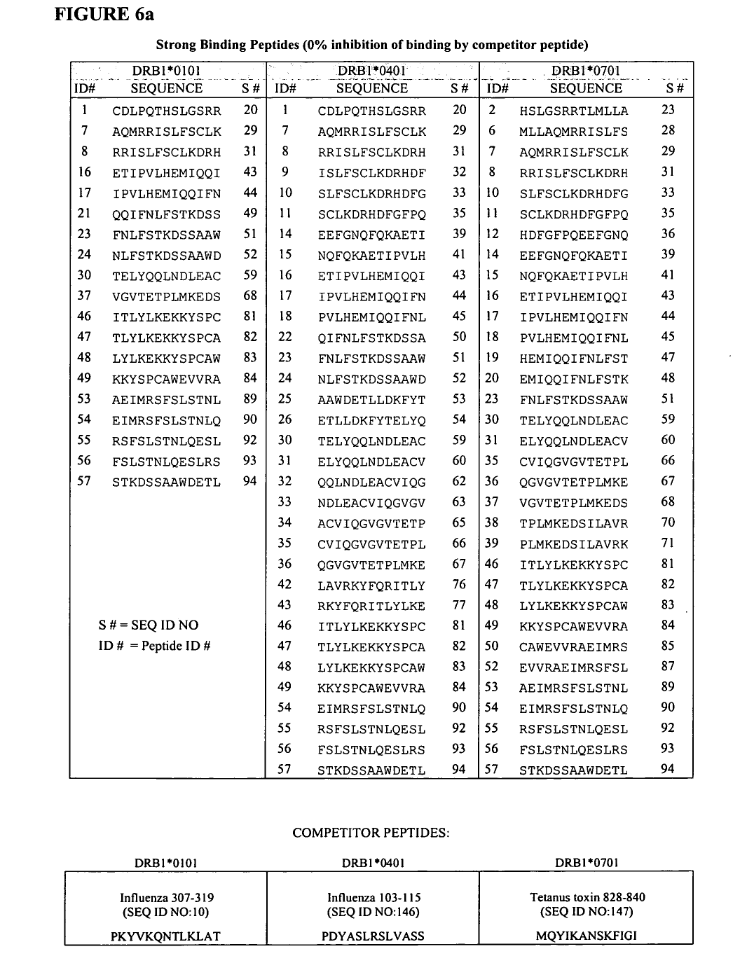

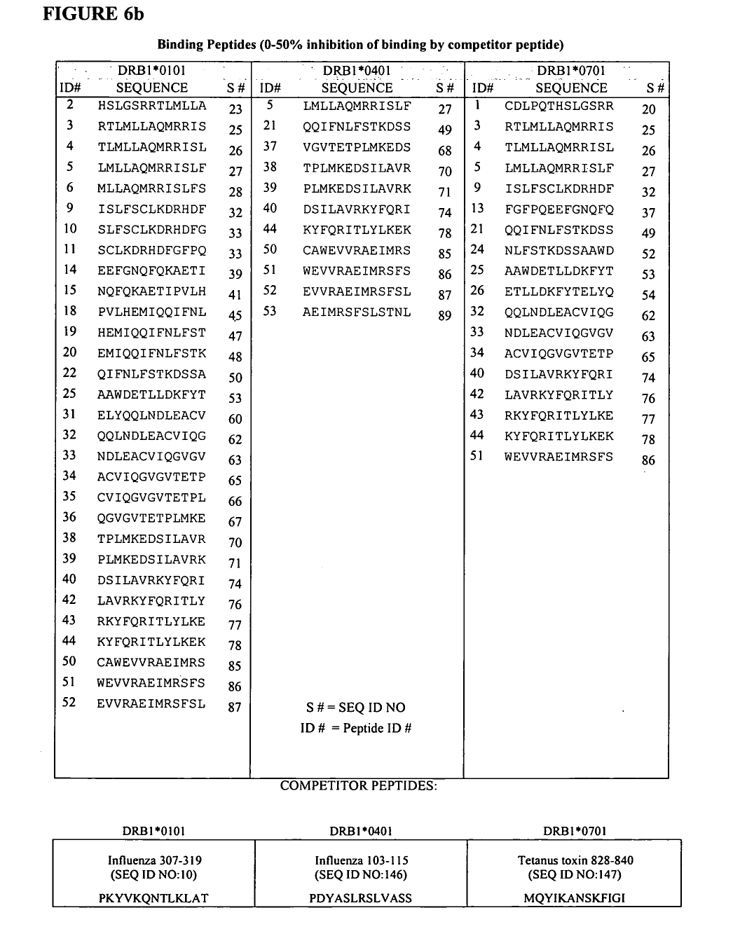

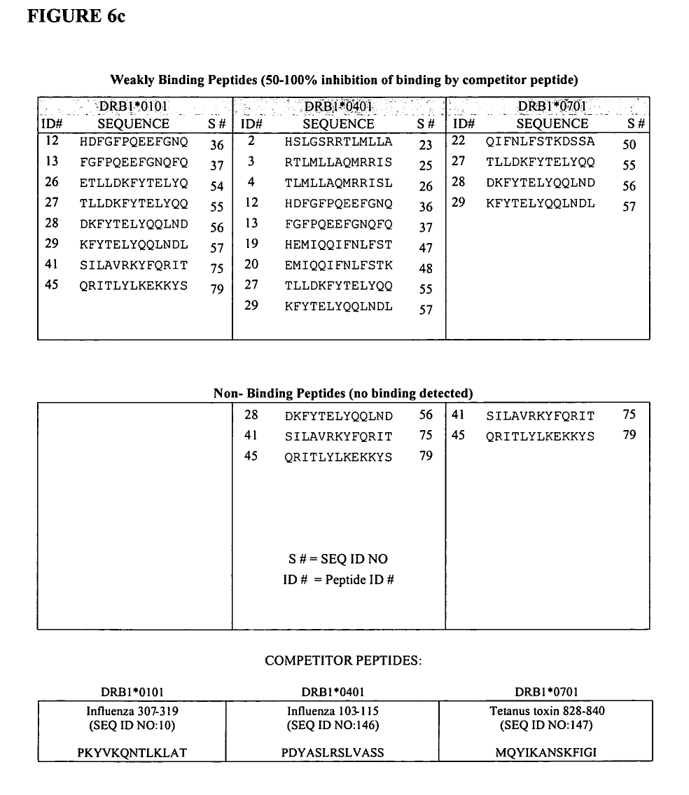

The binding assays conducted on the panel of 13-mer peptides as listed in FIG. 5 are depicted in FIG. 6a-d. With the exception of peptides shown in FIG. 6d, all peptides indicate a binding interaction with one or more of the human MHC class II allotypes tested.

### EXAMPLE 3

The interaction between MHC, peptide and T-cell receptor (TCR) provides the structural basis for the antigen specificity of T-cell recognition. T-cell proliferation assays test the binding of peptides to MHC and the recognition of MHC/peptide complexes by the TCR. In vitro T-cell proliferation assays of the present example, involve the stimulation of peripheral blood mononuclear cells (PBMCs), containing antigen presenting cells (APCs) and T-cells. Stimulation is conducted in vitro using synthetic peptide antigens, and in some experiments whole protein antigen. Stimulated T-cell proliferation is measured using ³H-thymidine (³H-Thy) and the presence of incorporated ³H-Thy assessed using scintillation counting of washed fixed cells.

Buffy coats from human blood stored for less than 12 hours were obtained from the National Blood Service (Addenbrooks Hospital, Cambridge, UK). Ficoll-paque was obtained from Amersham Pharmacia Biotech (Amersham, UK). Serum free AIM V media for the culture of primary human lymphocytes and containing L-glutamine, 50 82 g/ml streptomycin, 10 μg/ml gentomycin and 0.1% human serum albumin was from Gibco-BRL (Paisley, UK). Synthetic peptides were obtained from Eurosequence (Groningen, The Netherlands) and Babraham Technix (Cambridge, UK).

Erythrocytes and leukocytes were separated from plasma and platelets by gentle centrifugation of buffy coats. The top phase (containing plasma and platelets) was removed and discarded. Erythrocytes and leukocytes were diluted 1:1 in phosphate buffered saline (PBS) before layering onto 15 ml ficoll-paque (Amersham Pharmacia, Amersham UK). Centrifugation was done according to the manufacturers recommended conditions PBMCs were harvested from the serum+PBS/ficoll paque interface. PBMCs were mixed with PBS (1:1) and collected by centrifugation. The supernatant was removed and discarded and the PBMC pellet resuspended in 50 ml PBS. Cells were again pelleted by centrifugation and the PBS supernatant discarded. Cells were resuspended using 50 ml AIM V media and at this point counted and viability assessed using trypan blue dye exclusion. Cells were again collected by centrifugation and the supernatant discarded. Cells were resuspended for cryogenic storage at a density of 3×10⁷ per ml. The storage medium was 90% (v/v) heat inactivated AB human serum (Sigma, Poole, UK) and 10% (v/v) DMSO (Sigma, Poole, UK). Cells were transferred to a regulated freezing container (Sigma) and placed at −70° C. overnight. When required for use, cells were thawed rapidly in a water bath at 37° C. before transferring to 10 ml pre-warmed AIM V medium.

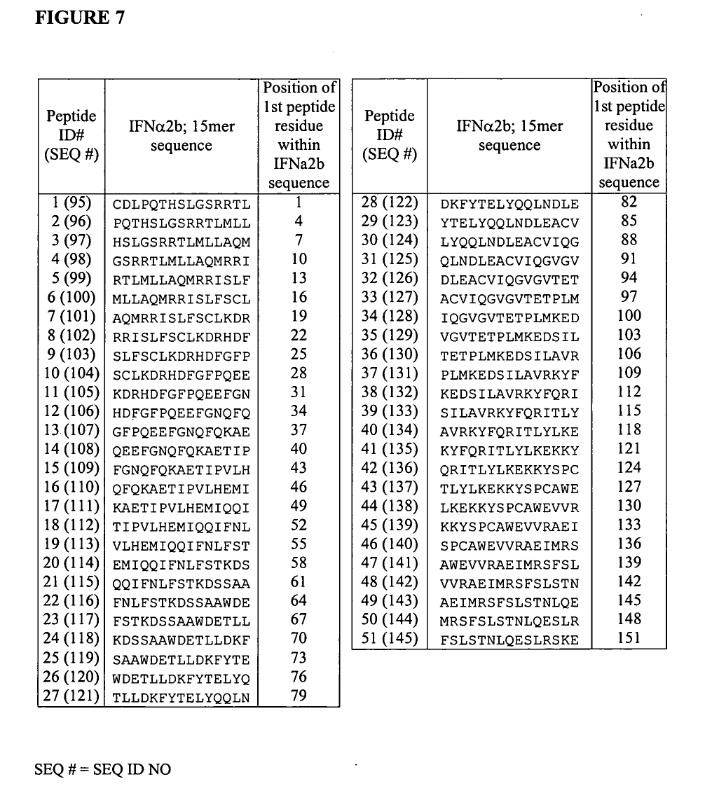

PBMC were stimulated with protein and peptide antigens in a 96 well flat bottom plate at a density of 2×10⁵ PBMC per well. PBMC were incubated for 7 days at 37° C. before pulsing with ³H-Thy (Amersham-Phamacia, Amersham, UK). For the present study, synthetic peptides (15 mers) that overlapped by 3aa increments were generated that spanned the entire sequence of IFNα. Peptide identification numbers (ID#) and sequences are given in FIG. 7. Each peptide was screened individually against PBMC's isolated from 20 naïve donors. Two control peptides that have previously been shown to be immunogenic and a potent non-recall antigen KLH were used in each donor assay.

The control antigens used in this study were as below:

| Peptide | Sequence |
|---|---|
| C-32 | Biotin-PKYVKQNTLKLAT |
| | Flu haemagglutinin 307-319 |
| C-49 | KVVDQIKKISKPVQH |
| | Chlamydia HSP 60 peptide |
| KLH | Whole protein from Keyhole |
| | Limpet Hemocyanin. |

Peptides were dissolved in DMSO to a final concentration of 10 mM, these stock solutions were then diluted 1/500 in AIM V media (final concentration 20 μM). Peptides were added to a flat bottom 96 well plate to give a final concentration of 2 and 20 μM in a 100 μl. The viability of thawed PBMC's was assessed by trypan blue-dye exclusion, cells were then resuspended at a density of 2×10⁶ cells/ml, and 100 μl (2×10⁵ PBMC/well) was transferred to each well containing peptides. Triplicate well cultures were assayed at each peptide concentration. Plates were incubated for 7 days in a humidified atmosphere of 5% CO₂ at 37° C. Cells were pulsed for 18-21 hours with 1 μCi ³H-Thy/well before harvesting onto filter mats. CPM values were determined using a Wallac microplate beta top plate counter (Perkin Elmer). Results were expressed as stimulation indices, determined using the following formula:
Proliferation to test peptide CPM/Proliferation in untreated wells CPM

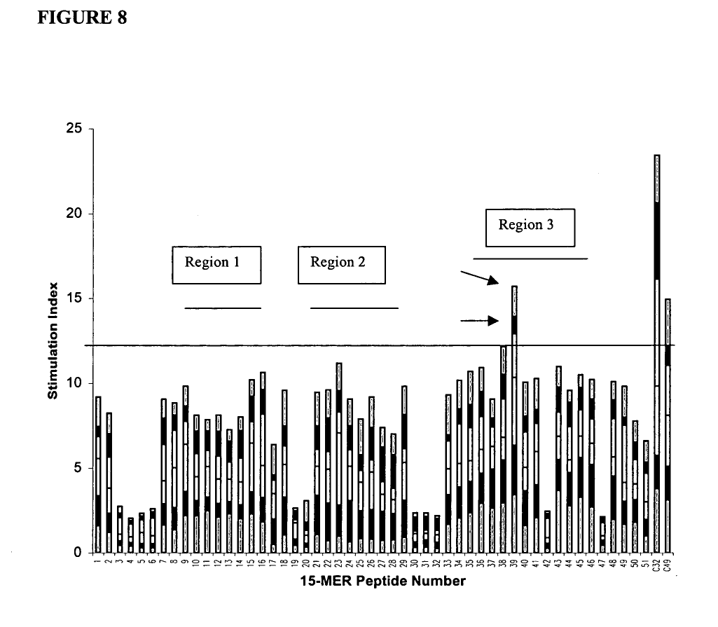

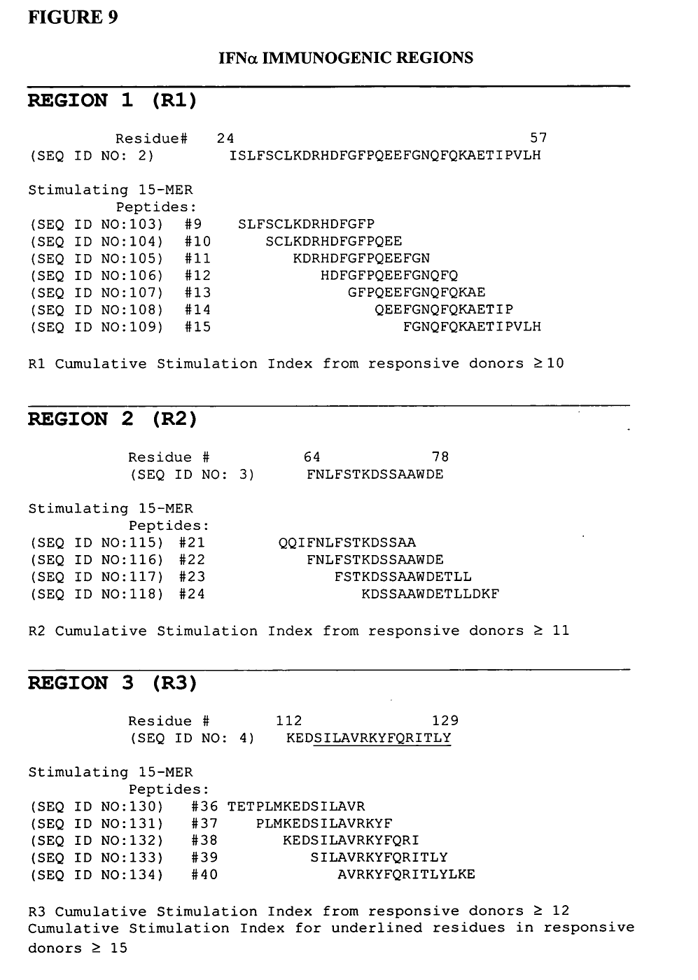

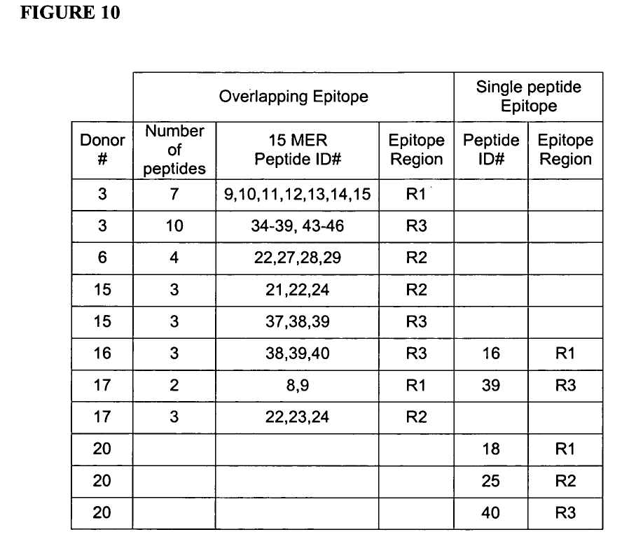

A stimulation index of 2 or greater was taken as positive stimulation in this assay. Mapping T cell epitopes in the IFNα sequence using the T cell proliferation assay resulted in the identification of three immunogenic regions R1, R2, R3. This was determined by T cell proliferation in 6 donors that responded to peptides in one or more of these regions. Region 3 is considered to contain a potential immunodominant T-cell epitope as proliferation is scored in 4 of 6 donors that responded to IFNα peptides. Regions 1 and 2 induce T-cell proliferation in certain individuals. The cumulative response data for the responding individuals is depicted in FIG. 8, and data from individual responders is summarized in FIG. 9. The stimulation index for individual donors is shown in FIG. 15. The epitope data for INFα and indicating R1, R2 and R3 together with the individual peptide/donor responses is depicted in FIG. 10.

### EXAMPLE 4

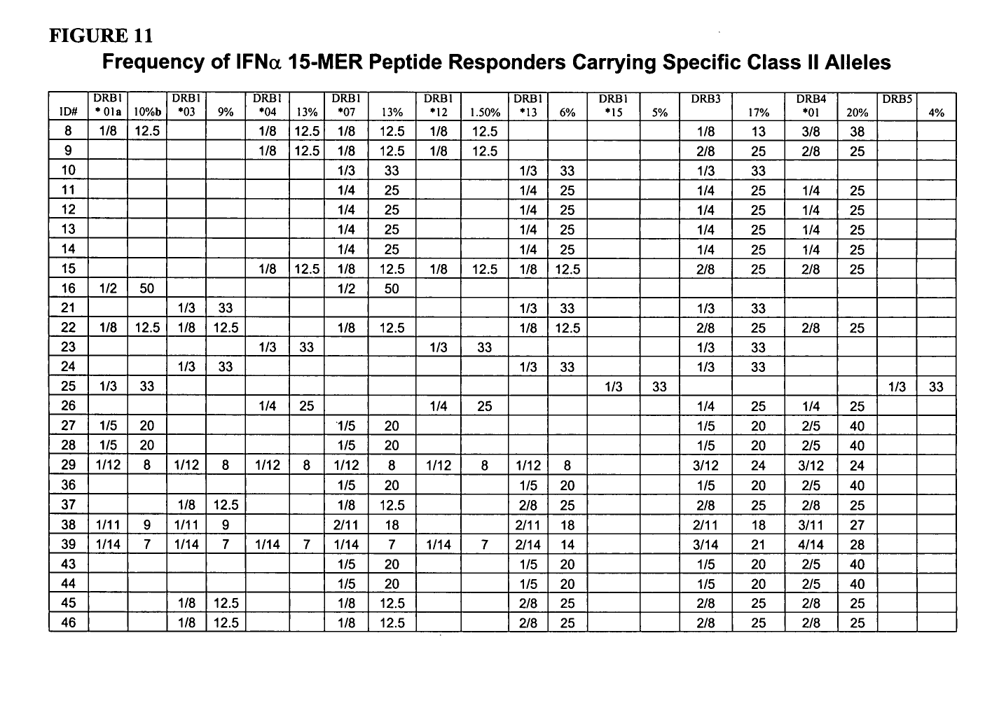

The tissue types for all PBMC samples used in EXAMPLE 3 were assayed using a commercially available reagent system (Dynal, Wirral, UK). Assays were conducted in accordance with the suppliers recommended protocols and standard ancillary reagents and agarose electrophoresis systems. Allotypic coverage for DRB1 alleles was 70% in the 20 donors tested. Results of the tissue typing were used to assess the frequency of INFα2 peptide responders carrying specific MHC class II alleles. Allotypic restriction of a given peptide is determined by the frequency of an allele in the donor population and the number of responding donors that express the same allele. If a peptide is associated with any particular allele then the frequency (expressed as a percentage) is expected to be greater than the frequency of the allele in the population. Results of such an analysis is given in FIG. 11. In general the small numbers preclude rigorous statistical examination, however the data indicate a possible association of peptides from the epitope region defined as R3 with the DRB4*01 allotype

### EXAMPLE 5

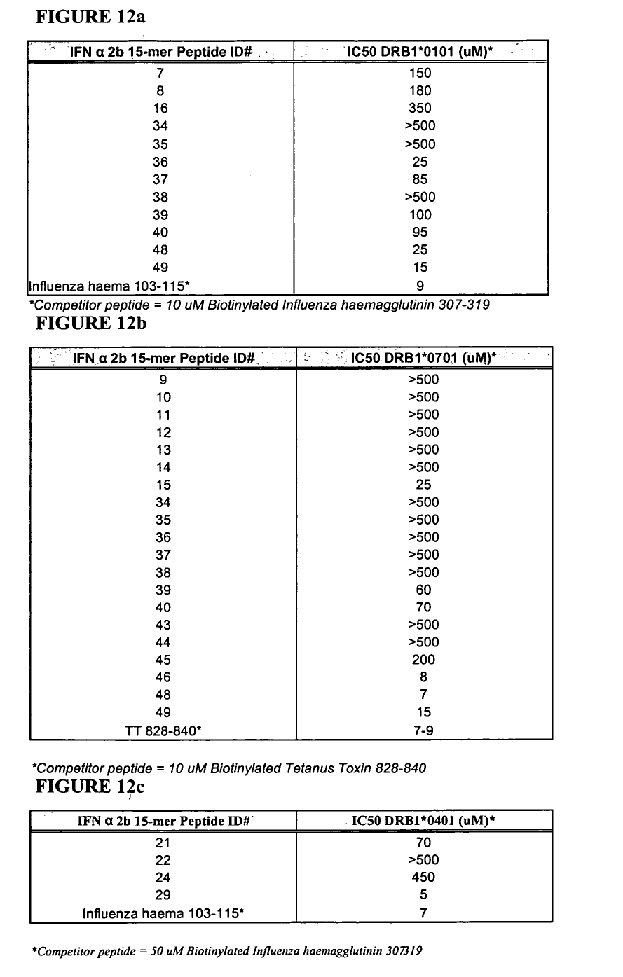

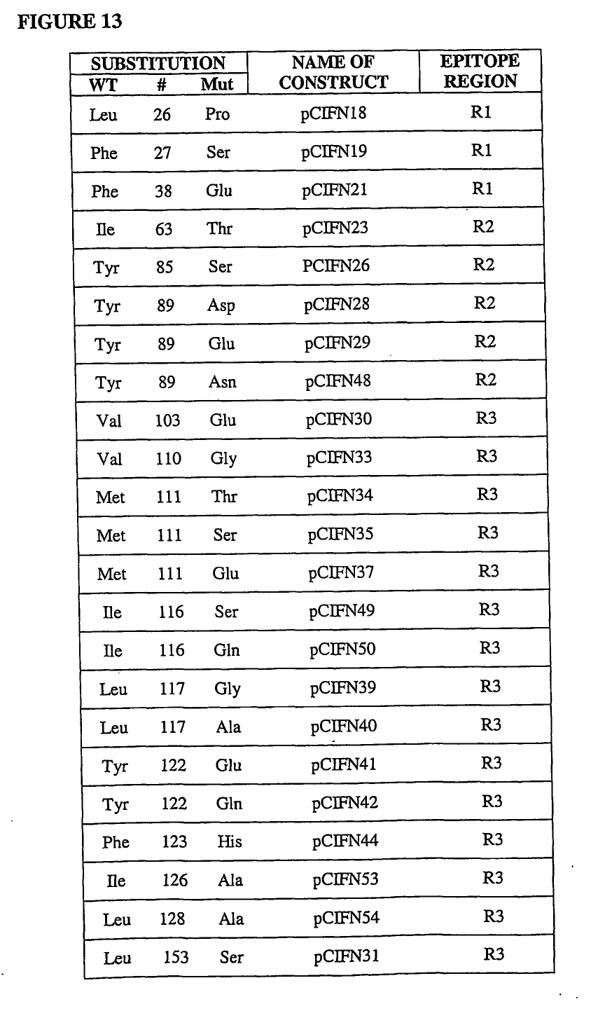

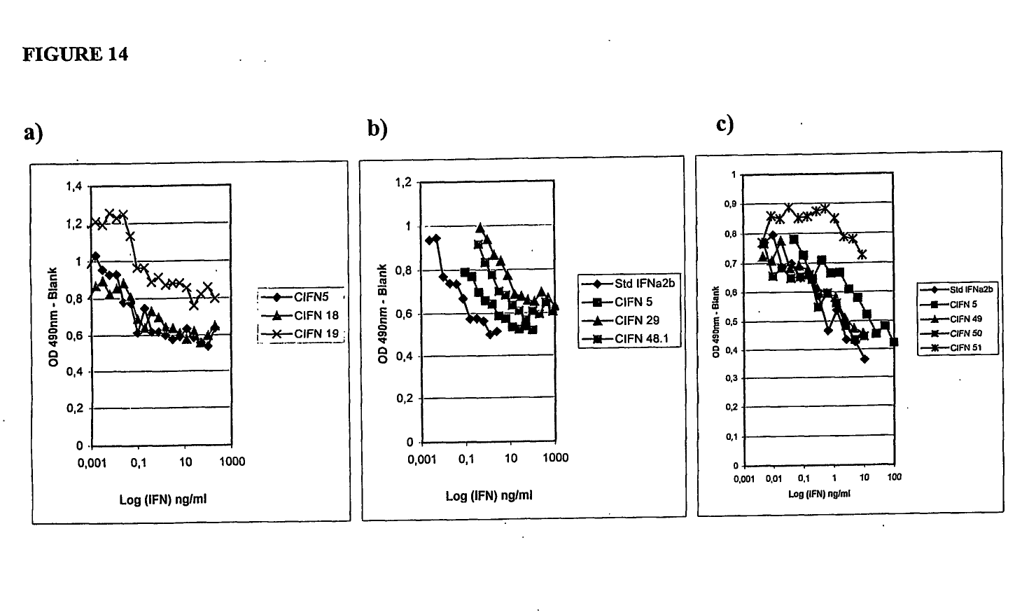

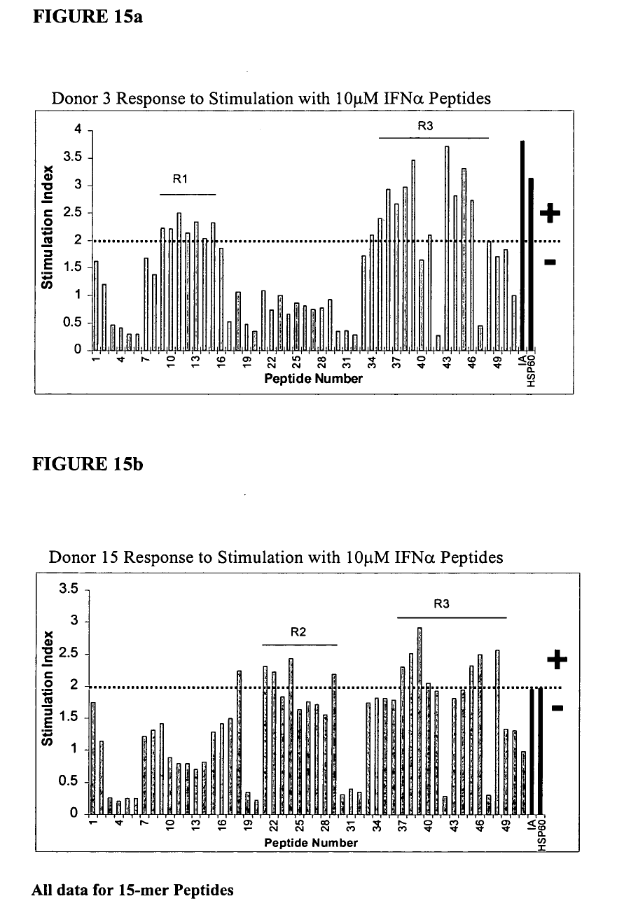

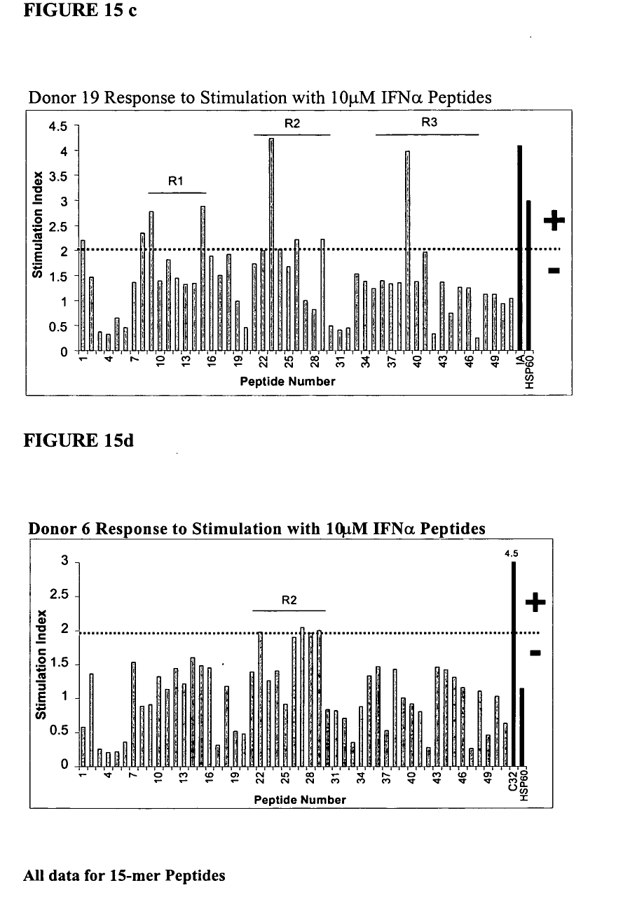

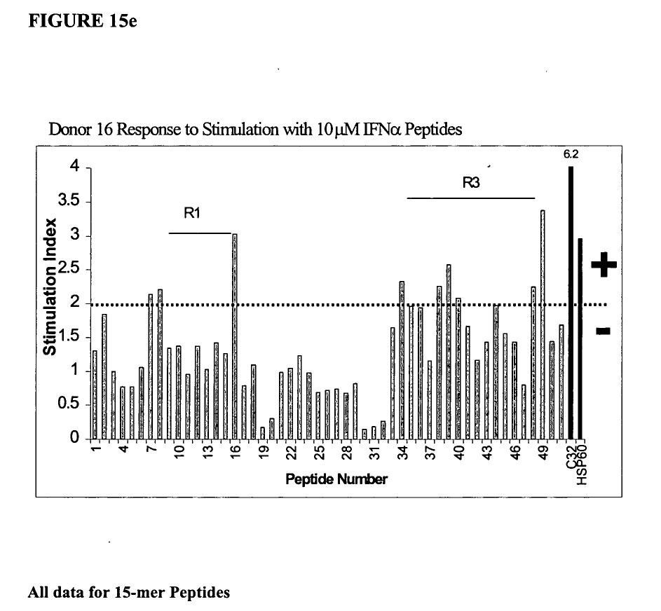

MHC Peptide binding assays were conducted using synthetic peptides containing sequences derived from the major immunogenic regions identified using the biological assay of EXAMPLE 3. In these assays synthetic 15-mer peptides were tested for their ability to bind three MHC allotypes in competition with a biotinylated competitor peptide. Assays were conducted broadly as detailed in EXAMPLE 2 and IC₅₀ values calculated from binding curves derived from six concentration ratios of competitor to test peptide. The IC₅₀ values for each peptide/allotype combination tested are shown in FIG. 12a-12c. These data indicate that peptides capable of stimulating T-cell proliferation in an in vitro biological assay may be of low or high affinity MHC class II ligands.

### EXAMPLE 6

A number of modified IFNα2 molecules were made using conventional recombinant DNA techniques. A wild-type INFα2b gene was cloned from human placental DNA and the gene was used both as a control reagent, and a template from which to derive modified INFα2b genes by site-directed mutagenesis. Wild-type and modified genes were inserted into a eukaryotic expression vector and the recombinant INFα2 proteins expressed as fusion protein with the human immunoglobulin constant region domain. Recombinant proteins were prepared from transiently transfected human embryonic kidney cells and assayed as detailed in EXAMPLE 7

Briefly, the wild-type INFα2b gene was amplified from human placental DNA (Sigma, Poole, UK) using the polymerase chain reaction (PCR). The gene contains no introns and was readily amplified using forward and reverse primers OL177 and OL178 containing restriction sites to facilitate cloning as given below:

OL177(EcoRI)
5′CCGGAATTCGCTAGCTGCCCAGCCGGCGATGGCCTGTGATCTGCCTCA
AACCCACAGCC-3′
OL178 (XhoI/BamHI)
5′-CCGGGATCCCTCGAGCTATTATTCCTTACTTCTTAAACTTTCTTGCA
AG-3′

The PCR product of 550 bp was digested with EcoRI and BamHI and cloned into the pLITMUS28 vector (NEB, LUK Ltd.). The sequence was confirmed to be that of interferon alpha 2b by analysis of a number of positive clones. In order to obtain expression from human embryonic kidney cells, the wild-type gene was re-cloned into vector pd-Cs [Lo, et al (1998), Protein Engineering 11: 495]. The pd-Cs vector directs the expression of a fusion protein containing the human immunoglobulin constant region domain. Cloning to this vector was achieved using PCR and primers OL232 and OL178. These primers provide cloning sites for use with enzymes XmaI and BamHI as below:

OL232 (XmaI)
5′ CTGTCCCCGGGTAAATGTGATCTGCCTCAGACCCACAGCC 3′
OL178 (XhoI/BamHI)
5′-CCGGGATCCCTCGAGCTATTATTCCTTACTTCTTAAACTTTCTTGCA
AG-3′

The PCR product of 530 bp was digested with XmaI and BamHI, purified using a Qiagen gel extraction kit and transferred into prepared pd-Cs from which the IFN(L) sequence had been removed using XmaI and BamHI. A positive clone was selected and the INFα2b sequence confirmed by sequence analysis. The pd-Cs vector containing the wild-type INFα2b gene was termed pCIFN5.

Single or multiple codon mutations to generate modified INFα2 genes is conducted by mutagenic PCR using pCIFN5 as a template. Overlap PCR was used to combine the two mutated halves of the interferon sequence. This fragment is then cloned into an intermediate vector (pGEM-T EASY vector; Promega, UK) for sequence analysis prior to being transferred into the pd-Cs derived expression vector using XmaI and BamHI as described above.

Mutagenesis was conducted using flanking primers OL235 and OL234 in separate reactions in combination with specific mutagenic (mis-matched) primers and the pCIFN5 template DNA.

| | |
|---|---|
| OL234: | 5′-CTCATGCTCCGTGATGCATGAGGC |
| OL235: | 5′-CACTGCATTCTAGTTGTGGTTTGTC |

Reactions were conducted using Expand IH Fidelity PCR reagents (Roche,GmbH) and reaction conditions specified by the following cycle:
94° C./2′+25 Cycles@94° C./30″, 60° C./30″, 72° C./30″+72° C./10″

The products of the separate reactions were joined by PCR in a reaction driven by primers OL235 and OL234 using 15 cycles of PCR as above.

PCR products were gel purified using commercially available kit systems (Qiagen gel extraction kit). The products were cloned using a T/A cloning system into vector pGEM-T EASY (Promega, UK) and a number of clones were sequenced in each case to confirm the successful introduction of the desired mutation.

The desired clones were digested with BamH1 and Xma1 and the purified product ligated into a prepared pd-Cs vector. Cloning was conducted using E. coli XL1-Blue cells (Strategene Europe) and culture conditions recommended by the supplier. Sequence confirmation was conducted on all final vector preparations using OL261 and OL234 as sequencing primers.

| | |
|---|---|
| OL261 | 5′-GGTGACAGAGACTCCCCTGATGAAG 3′ |
| OL234: | 5′-CTCATGCTCCGTGATGCATGAGGC 3′ |

Expression of modified INFα2 human IgFC fusion proteins was achieved using HEK293 human embryonic kidney cell line as the expression host. All DNA for transfection was prepared using the high purity CONCERT midiprep system and instructions provided by the supplier (Invitrogen, Paisley, UK). DNA is filter sterilised prior to use and quantified by measurment of the A₂₆₀. Concentrations were adjusted to 0.5-1.0 μg/μl.

For transient expression, HEK293 were grown using D-MEM glutamax medium (Invitrogen, Paisley, UK) supplemented with 10% FCS and 250 μg/ml geneticin. Prior to transfection, cells were collected by treatment with trypsin and washed using PBS. After 2 cycles of washing cells are taken into fresh medium at a density of 4×10⁵ cells/ml, and plated into multiwell dishes pre-treated with poly-l-lysine to ensure good cell adhesion. Typically, 2×10⁵ cells are added to each well of a 48 well plate and the plates incubated overnight at 37° C./5% CO₂.

Prior to transfection, the medium is replaced in each well and the transfection mixes added. Transfection is conducted using the lipofectamine reagent and instructions provided by the supplier (Invitrogen, Paisley, UK). Briefly, transfection mixes are prepared containing lipofectamine, OPTI-MEM (Invitrogen, Paisley, UK) and 0.8 μg DNA per well for each expression vector construct. Transfection mixes are added to the cells and the cells incubated for 4-6 hours. The medium is replaced with 0.5 ml fresh media and the cells incubated at 37° C./5% CO₂. Samples were taken after 48 hours for analysis by both anti-FC ELISA and Daudi cell proliferation assay. The media was harvested after 7 days and stored at 4 C for further analysis as above.

The medium is assayed for the presence of INFα2 using a commercially available ELISA system and instructions provided by the supplier (R&D systems, UK). In some instances an ELISA detecting the human immunglobulin constant region domain of the IFNα-fusion protein was applied. For this assay a mouse anti-human IgG Fc preparation (Sigma, Poole, UK) is used as a capture reagent. The INFa-HuFc fusion is quantitated with reference to a standard curve generated using a dilution series of a reference human IgG preparation (Sigma). Bound INFα-FC fusion or the reference protein is detected using an anti-human IgG peroxidase conjugate (Sigma) and Sigma OPD colourimetric substrate.

Following estimation of the amount of INFα in the HEK293 conditioned medium, the conditioned medium is used directly to test the functional activity of the modified INFα using the anti-proliferation assay as detailed in EXAMPLE 7.

### EXAMPLE 7

Modified interferon molecules of the present invention were tested for their ability to inhibit the growth of human B cell lymphoma line Daudi. The method is broadly as described previously [Mark, D. F. et al (1984) Proc. Natl. Acad. Sci. USA 81: 5662-5666] and involves incubation of Daudi cells with the test interferon. The anti-proliferative effect of the test molecule is measured using a soluble dye substance which undergoes a colour change in the presence of proliferating cells. The induced colour change is measured in a spectrophotometer and any antiproliferative effect is computed with reference to the colour change recorded in non-treated control cells and cells treated with a standard interferon preparation.

Briefly, Daudi cells (ATCC # CCL-213) were cultured RPMI 1640 Media supplemented with 100 units/ml Penicillin/100 ug/ml Streptomycin and 2 mM L-Glutamine and 20% Fetal Bovine Serum (FBS). All media and supplements were from Gibco (Paisley, UK). The day before assay, cells are replaced into fresh medium at a density 0.9×10⁶/ml and next day replaced into fresh medium as above except containing 10% (v/v) FBS. The cell density is adjusted to be 2×10⁵ cells/ml.

The test and control interferon preparations are diluted into RPMI containing 10% FBS. Dilutions are made into 96-well flat bottom plates to contain 100 ul/well and all samples are set up in triplicate. Typically doubling dilution series are set out across each plate. Positive control wells are also included in triplicate with a starting concentration of the interferon standard (NIBSC, South Mimms, UK) at 10000 pg/ml. Control wells containing 100 ul media alone (no interferon) are also included. 100 ul of the cells are added to each well, and the plates incubated for 72 hours at 37° C., 5% CO₂.

Proliferation is assessed using Aqueous One reagent system and the suppliers recommended protocol (Promega, Southampton, UK). Briefly, 40 μl of the Aqueous One reagent is added to all wells and the substrate mixed. Plates are incubated at 37° C. for one hour, and then transferred to the plate reading instrument for determination of the light absorbance. Readings are taken at 490 nm. Average absorbance at 490 nm is plotted on the Y axis versus concentrations of interferon standard added along the X axis. Interferon concentration is determined using a ELISA techniques as detailed in EXAMPLE 6. The resulting calibration curve is used to determine the ED₅₀ value for each test sample.

Results of such an analysis according to the above method for a number of modified INFα2 molecules are depicted in FIG. 14. The results indicate retained anti-proliferative properties in the presence of amino acid substitutions within the INFα2 sequence.

1. A modified molecule having the biological activity of human interferon alpha 2 (INFα2) and being substantially non-immunogenic or less immunogenic than any non-modified molecule having the same biological activity when used in vivo.

2. A molecule according to claim 1, wherein said loss of immunogenicity is achieved by removing one or more T-cell epitopes derived from the originally non-modified molecule and/or by reduction in numbers of MHC allotypes able to bind peptides derived from said molecule.

3. A molecule according to claim 2, wherein one T-cell epitope is removed.

4-70. (canceled)

71. A protein that is homologous to human interferon alpha 2 (INFα2), the human interferon alpha 2 having an amino acid sequence (SEQ ID NO: 1) that includes at least one T-cell epitope;
the protein having substantially the same amino acid sequence as SEQ ID NO: 1, but including at least one less T-cell epitope;
wherein the protein has substantially the same biological activity as human interferon alpha 2, but is less immunogenic than said human interferon alpha 2 when both are exposed to the immune system of the same species.

72. The protein of claim 71 wherein the amino acid sequence of the protein includes one less T-cell epitope.

73. The protein of claim 71 wherein the amino acid sequence of the protein differs from SEQ ID NO: 1 by one to nine amino acid residues.

74. The protein of claim 71 wherein the amino acid sequence of the protein has at least one less amino acid residue than SEQ ID NO: 1.

75. The protein of claim 71 wherein the amino acid sequence of the protein has at least one more amino acid residue than SEQ ID NO: 1.

76. The protein of claim 71 wherein the amino acid sequence of the protein has the same number of amino acid residues as SEQ ID NO: 1.

77. A pharmaceutical composition comprising a protein of claim 71 and a pharmaceutically acceptable carrier therefor.

78. A method of preparing a protein of claim 71, the method comprising the steps of:
(i) identifying one or more potential T-cell epitopes within the amino acid sequence of human interferon alpha 2 (SEQ ID NO: 1);
(ii) selecting at least one sequence variant of at least one potential T-cell epitope identified in step (i) that eliminates or substantially reduces the MHC class II binding activity of the potential T-cell epitope; wherein the amino acid sequence of the selected variant differs from the amino acid sequence of the T-cell epitope identified in step (i) by at least one amino acid residue;
(iii) preparing, by recombinant DNA techniques, at least one protein that includes at least one variant selected in step (ii);
(iv) evaluating the biological activity and immunogenicity of at least one protein prepared in step (iii); and
(v) selecting a protein evaluated in step (iv) that has substantially the same biological activity as, but substantially less immunogenicity than human interferon alpha 2.

79. The method of claim 78 wherein step (i) is carried out by determining the MHC class II binding affinity of potential T-cell epitope segments of human interferon alpha 2 using an in vitro assay, an in silico technique, or a biological assay.

80. The method of claim 78 wherein step (i) is carried out by:
(a) selecting a region of the amino acid sequence of human interferon alpha 2 (SEQ ID NO: 1);
(b) sequentially sampling overlapping amino acid residue segments of predetermined uniform size and including at least three amino acid residues from the selected region;
(c) calculating the MHC class II molecule binding score for each of the sampled segments by summing assigned values for each hydrophobic amino acid residue side chain present in the sampled amino acid residue segment; and
(d) identifying at least one segment that is suitable for modification based on the calculated MHC class II binding score for that segment to reduce the overall MHC class II binding score for the protein relative to the binding score for human interferon alpha 2.

81. The method of claim 80 wherein step (c) is carried out by using a Böhm scoring function modified to include a van der Waal's ligand-protein energy repulsive term and a ligand conformational energy term by:
(1) selecting a model from a first database of MHC class II molecule models;
(2) selecting an allowed peptide backbone from a second database of allowed peptide backbones for the MHC class II molecule models in step (1);
(3) identifying amino acid residue side chains present in each sampled segment;
(4) determining the binding affinity value for all side chains present in each sampled segment; and
(5) repeating each of steps (1) through (4) for each model in the first database and for each backbone in the second database.

82. The method of claim 78 wherein step (ii) is carried out by substitution, addition, or deletion of one to nine amino acid residues from a potential T-cell epitope identified in step (i).

83. A protein of claim 71 having an amino acid sequence that is free from T-cell epitopes.

84. A protein having the following amino acid sequence (SEQ ID NO: 5): CDLPQTHSLGSRRTLMLLAQMRX⁰ISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKX¹X²QRX³TX4YLKEKKYSPCAWEVVRAEIMRSFSLSTNLQESLRSKE,
wherein X⁰ is R or K; X¹ is Y, E, or Q; X² is F or H; X³ is I or A; and X⁴ is L or A;
excluding proteins having amino acid sequences in which, simultaneously, X¹ is Y, X² is F, X³ is I, and X⁴ is L.

85. A protein having the following amino acid sequence (SEQ ID NO: 6): CDLPQTHSLGSRRTLMLLAQMRX⁰ISLFSCLKDRHDFGFPQEEFGNQFQKAETIPVLHEMIQQIFNLFSTKDSSAAWDETLLDKFYTELYQQLNDLEACVIQGVGVTETPX¹X2KEDSX³X⁴AVRKX⁵X⁶QRX⁷TX⁸YLKEKKYSPCAWEVVRAEIMRSFSX⁹STNLQESLRSKE,
wherein X⁰ is R or K; X¹ is L, S, or G; X² is M, T, S, or E; X³ is I, S, or Q; X⁴ is L or G; X⁵ is Y, E, or Q; X⁶ is F or H; X⁷ is I or A; X⁸ is L or A; and X⁹ is L or S;
excluding proteins having amino acid sequences in which, simultaneously, X¹ is L; X² is M; X³ is; X⁴ is L; X⁵ is Y; X⁶ is F; X⁷ is I; X⁸ is L; and X⁹ is L.

86. A protein having the following amino acid sequence (SEQ ID NO: 7): CDLPQTHSLGSRRTLMLLAQMRX0ISLFSCLKDRHDFGFPQEEFGNQFQKAETIPVLHEMIQQX1X2NX3X4STKDSSAAX5DETLLDKX6X7TELX8QQLNDLEACVIQGVGVTETPLMKEDSILAVRKYFQRITLYLKEKKYSPCAWEVVRAEIMRSFSLSTNLQESLRSKE,
wherein X⁰ is R or K; X¹ is 1 or T; X² is F, D, or A; X³ is L or A; X⁴ is F, D, or E; X⁵ is W or H; X⁶ is F, D, or E; X⁷ is Y or S; and X⁸ is Y, D, E, or N;
excluding proteins having amino acid sequences in which, simultaneously, X¹ is I; X² is F; X³ is L; X⁴is F; X⁵ is W; X⁶ is F; X⁷ is Y; and X⁸ is Y.

87. A protein having the following amino acid sequence (SEQ ID NO: 8): CDLPQTHSLGSRRTLMLLAQMRX⁰ISLFSCLKDRHDFGFPQEEFGNQFQKAETIPVLHEMIQQX¹FNLFSTKDSSAAWDETLLDKFX²TELX³QQLNDLEACVIQGVGVTETPLMKEDSILAVRKYFQRITLYLKEKKYSPCAWEVVRAEIMRSFSLSTNLQESLRSKE,
wherein X⁰ is R or K; X¹ is I or T; X² is Y or S and X³ is Y, D, E, or N;
excluding proteins having amino acid sequences in which, simultaneously, X¹ is I; X² is Y; and X³ is Y.

88. A protein having the following amino acid sequence (SEQ ID NO: 9): CDLPQTHSLGSRRTLMLLAQMRX⁰ISX¹X²SCLKDRHDFGX³PQEEFGNQFQKAETIPX⁴LHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE,
wherein X⁰ is R or K; X¹ is L or P; X² is F or S; X³ is F or E; and X⁴ is V or A;
excluding proteins having amino acid sequences in which, simultaneously, X¹ is L; X² is F; X³ is F; and X⁴ is V.

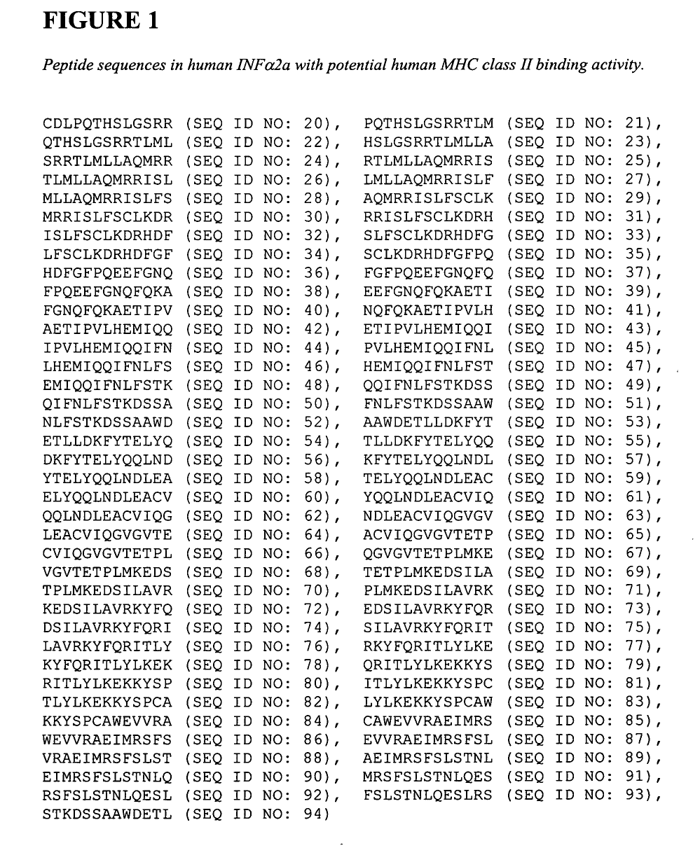

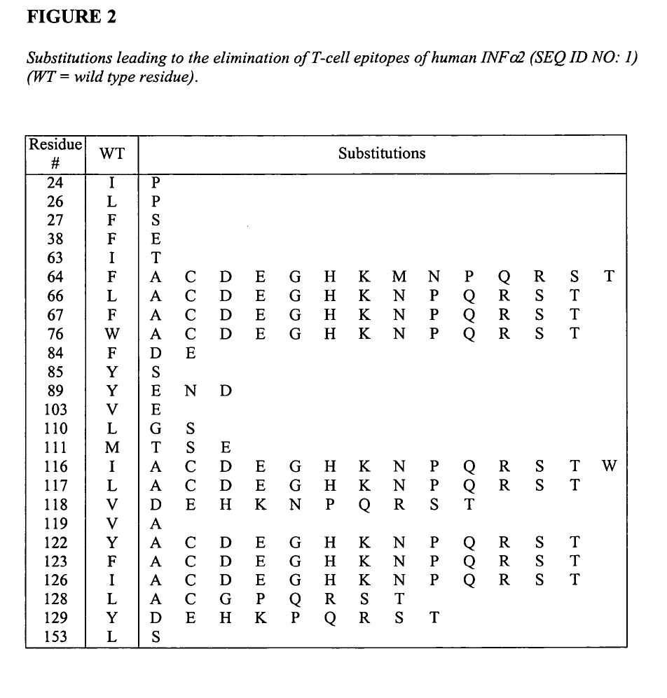

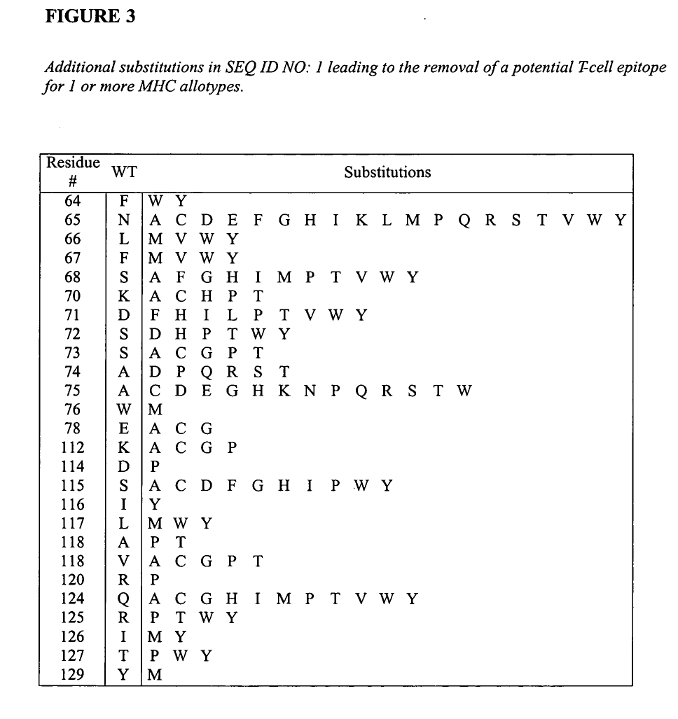

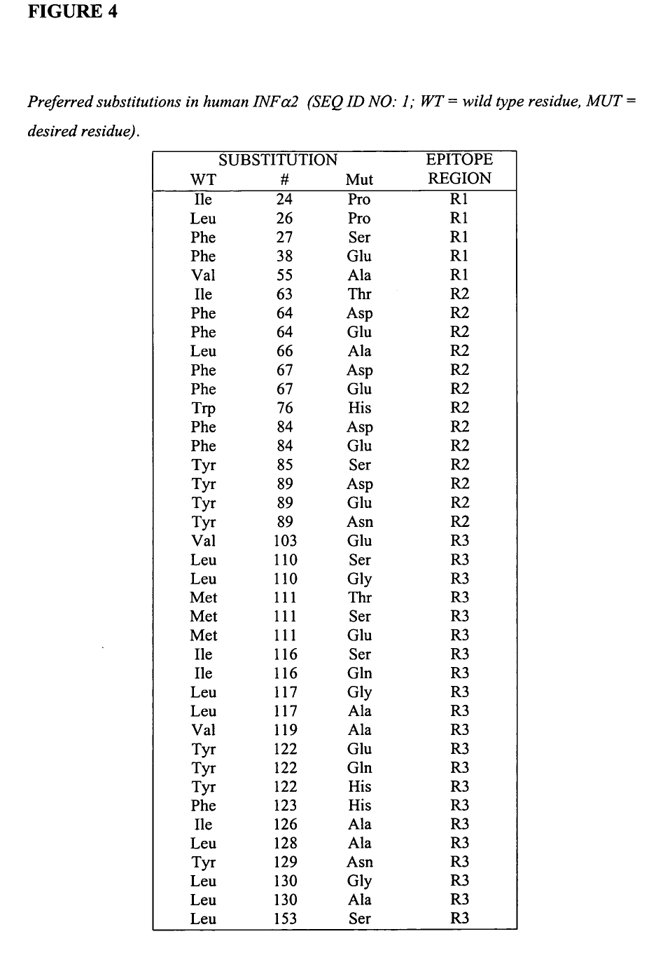

89. An isolated polypeptide selected from the group of polypeptides set forth in FIG. 1.

90. An isolated polypeptide selected from the group of polypeptides set forth in FIG. 7.

91. An isolated polynucleotide encoding a protein of claim 71.

92. An isolated polynucleotide encoding a protein of claim 84.

93. An isolated polynucleotide encoding a protein of claim 85.

94. An isolated polynucleotide encoding a protein of claim 86.

95. An isolated polynucleotide encoding a protein of claim 87.

96. An isolated polynucleotide encoding a protein of claim 88.

97. An isolated polynucleotide encoding a polypeptide of claim 89.

98. An isolated polynucleotide encoding a polypeptide of claim 90.

99. A plasmid comprising a polynucleotide of claim 91.

100. A plasmid comprising a polynucleotide of claim 92.

101. A plasmid comprising a polynucleotide of claim 93.

102. A plasmid comprising a polynucleotide of claim 94.

103. A plasmid comprising a polynucleotide of claim 95.

104. A plasmid comprising a polynucleotide of claim 96.

105. A plasmid comprising a polynucleotide of claim 97.

106. A plasmid comprising a polynucleotide of claim 98.


SEQUENCE LISTING (147 sequences)

SEQ ID NO: 1 - protein, 164 aa, Homo Sapiens
  VARIANT 23: Xaa = Arg, Lys
CDLPQTHSLGSRRTLMLLAQMRXSLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 2 - protein, 34 aa, Artificial Sequence
  HMC class II binding epitope
ISLFSCLKDRHDFGFPQEEFGNQFQKAETIPVLH

SEQ ID NO: 3 - protein, 15 aa, Artificial Sequence
  HMC class II binding epitope
FNLFSTKDSSAAWDE

SEQ ID NO: 4 - protein, 18 aa, Artificial Sequence
  HMC class II binding epitope
KEDSILAVRKYFQRITLY

SEQ ID NO: 5 - protein, 165 aa, Artificial Sequence
  Interferon variants
CDLPQTHSLGSRRTLMLLAQMRXISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKXXQRXTXYLKEKKYSPCAWEVVRAEIMRSFSLSTNLQESLRSKE

SEQ ID NO: 6 - protein, 165 aa, Artificial Sequence
  Interferon variants
CDLPQTHSLGSRRTLMLLAQMRXISLFSCLKDRHDFGFPQEEFGNQFQKAETIPVLHEMIQQIFNLFSTKDSSAAWDETLLDKFYTELYQQLNDLEACVIQGVGVTETPXXKEDSXXAVRKXXQRXTXYLKEKKYSPCAWEVVRAEIMRSFSXSTNLQESLRSKE

SEQ ID NO: 7 - protein, 165 aa, Artificial Sequence
  Interferon variants
CDLPQTHSLGSRRTLMLLAQMRXISLFSCLKDRHDFGFPQEEFGNQFQKAETIPVLHEMIQQXXNXXSTKDSSAAXDETLLDKXXTELXQQLNDLEACVIQGVGVTETPLMKEDSILAVRKYFQRITLYLKEKKYSPCAWEVVRAEIMRSFSLSTNLQESLRSKE

SEQ ID NO: 8 - protein, 165 aa, Artificial Sequence
  Interferon variants
CDLPQTHSLGSRRTLMLLAQMRXISLFSCLKDRHDFGFPQEEFGNQFQKAETIPVLHEMIQQXFNLFSTKDSSAAWDETLLDKFXTELXQQLNDLEACVIQGVGVTETPLMKEDSILAVRKYFQRITLYLKEKKYSPCAWEVVRAEIMRSFSLSTNLQESLRSKE

SEQ ID NO: 9 - protein, 165 aa, Artificial Sequence
  Interferon variants
CDLPQTHSLGSRRTLMLLAQMRXISXXSCLKDRHDFGXPQEEFGNQFQKAETIPXLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 10 - protein, 13 aa, Artificial Sequence
  Antigens
PKYVKQNTLKLAT

SEQ ID NO: 11 - protein, 15 aa, Artificial Sequence
  Antigens
KVVDQIKKISKPVQH

SEQ ID NO: 12 - DNA, 59 bp, Artificial Sequence
  Primers
ccggaattcgctagctgcccagccggcgatggcctgtgatctgcctcaaacccacagcc

SEQ ID NO: 13 - DNA, 49 bp, Artificial Sequence
  Primers
ccgggatccctcgagctattattccttacttcttaaactttcttgcaag

SEQ ID NO: 14 - DNA, 40 bp, Artificial Sequence
  Primers
ctgtccccgggtaaatgtgatctgcctcagacccacagcc

SEQ ID NO: 15 - DNA, 49 bp, Artificial Sequence; identical to SEQ ID NO: 13
  Primers

SEQ ID NO: 16 - DNA, 24 bp, Artificial Sequence
  Primers
ctcatgctccgtgatgcatgaggc

SEQ ID NO: 17 - DNA, 25 bp, Artificial Sequence
  Primers
cactgcattctagttgtggtttgtc

SEQ ID NO: 18 - DNA, 25 bp, Artificial Sequence
  Primers
ggtgacagagactcccctgatgaag

SEQ ID NO: 19 - DNA, 24 bp, Artificial Sequence
  Primers
ctcatgctccgtgatgcatgaggc

SEQ ID NO: 20 - protein, 13 aa, Artificial Sequence
  MHC Class II binding epitope
CDLPQTHSLGSRR

SEQ ID NO: 21 - protein, 13 aa, Artificial Sequence
  MHC Class II binding epitope
PQTHSLGSRRTLM

SEQ ID NO: 22 - protein, 13 aa, Artificial Sequence
  MHC Class II binding epitope
QTHSLGSRRTLML

SEQ ID NO: 23 - protein, 13 aa, Artificial Sequence
  MHC Class II binding epitope
HSLGSRRTLMLLA

SEQ ID NO: 24 - protein, 13 aa, Artificial Sequence
  MHC Class II binding epitope
SRRTLMLLAQMRR

SEQ ID NO: 25 - protein, 13 aa, Artificial Sequence
  MHC Class II binding epitope
RTLMLLAQMRRIS

SEQ ID NO: 26 - protein, 13 aa, Artificial Sequence
  MHC Class II binding epitope
TLMLLAQMRRISL

SEQ ID NO: 27 - protein, 13 aa, Artificial Sequence
  MHC Class II binding epitope
LMLLAQMRRISLF

SEQ ID NO: 28 - protein, 13 aa, Artificial Sequence
  MHC Class II binding epitope
MLLAQMRRISLFS

SEQ ID NO: 29 - protein, 13 aa, Artificial Sequence
  MHC Class II binding epitope
AQMRRISLFSCLK

SEQ ID NO: 30 - protein, 13 aa, Artificial Sequence
  MHC Class II binding epitope
MRRISLFSCLKDR

SEQ ID NO: 31 - protein, 13 aa, Artificial Sequence
  MHC Class II binding epitope
RRISLFSCLKDRH

SEQ ID NO: 32 - protein, 13 aa, Artificial Sequence
  MHC Class II binding epitope
ISLFSCLKDRHDF

SEQ ID NO: 33 - protein, 13 aa, Artificial Sequence
  MHC Class II binding epitope
SLFSCLKDRHDFG

SEQ ID NO: 34 - protein, 13 aa, Artificial Sequence
  MHC Class II binding epitope
LFSCLKDRHDFGF

SEQ ID NO: 35 - protein, 13 aa, Artificial Sequence
  MHC Class II binding epitope
SCLKDRHDFGFPQ

SEQ ID NO: 36 - protein, 13 aa, Artificial Sequence
  MHC Class II binding epitope
HDFGFPQEEFGNQ

SEQ ID NO: 37 - protein, 13 aa, Artificial Sequence
  MHC Class II binding epitope
FGFPQEEFGNQFQ

SEQ ID NO: 38 - protein, 13 aa, Artificial Sequence
  MHC Class II binding epitope
FPQEEFGNQFQKA

SEQ ID NO: 39 - protein, 13 aa, Artificial Sequence
  MHC Class II binding epitope
EEFGNQFQKAETI

SEQ ID NO: 40 - protein, 13 aa, Artificial Sequence
  MHC Class II binding epitope
FGNQFQKAETIPV

SEQ ID NO: 41 - protein, 13 aa, Artificial Sequence
  MHC Class II binding epitope
NQFQKAETIPVLH

SEQ ID NO: 42 - protein, 13 aa, Artificial Sequence
  MHC Class II binding epitope
AETIPVLHEMIQQ

SEQ ID NO: 43 - protein, 13 aa, Artificial Sequence
  MHC Class II binding epitope
ETIPVLHEMIQQI

SEQ ID NO: 44 - protein, 13 aa, Artificial Sequence
  MHC Class II binding epitope
IPVLHEMIQQIFN

SEQ ID NO: 45 - protein, 13 aa, Artificial Sequence
  MHC Class II binding epitope
PVLHEMIQQIFNL

SEQ ID NO: 46 - protein, 13 aa, Artificial Sequence
  MHC Class II binding epitope
LHEMIQQIFNLFS

SEQ ID NO: 47 - protein, 13 aa, Artificial Sequence
  MHC Class II binding epitope
HEMIQQIFNLFST

SEQ ID NO: 48 - protein, 13 aa, Artificial Sequence
  MHC Class II binding epitope
EMIQQIFNLFSTK

SEQ ID NO: 49 - protein, 13 aa, Artificial Sequence
  MHC Class II binding epitope
QQIFNLFSTKDSS

SEQ ID NO: 50 - protein, 13 aa, Artificial Sequence
  MHC Class II binding epitope
QIFNLFSTKDSSA

SEQ ID NO: 51 - protein, 13 aa, Artificial Sequence
  MHC Class II binding epitope
FNLFSTKDSSAAW

SEQ ID NO: 52 - protein, 13 aa, Artificial Sequence
  MHC Class II binding epitope
NLFSTKDSSAAWD

SEQ ID NO: 53 - protein, 13 aa, Artificial Sequence
  MHC Class II binding epitope
AAWDETLLDKFYT

SEQ ID NO: 54 - protein, 13 aa, Artificial Sequence
  MHC Class II binding epitope
ETLLDKFYTELYQ

SEQ ID NO: 55 - protein, 13 aa, Artificial Sequence
  MHC Class II binding epitope
TLLDKFYTELYQQ

SEQ ID NO: 56 - protein, 13 aa, Artificial Sequence
  MHC Class II binding epitope
DKFYTELYQQLND

SEQ ID NO: 57 - protein, 13 aa, Artificial Sequence
  MHC Class II binding epitope
KFYTELYQQLNDL

SEQ ID NO: 58 - protein, 13 aa, Artificial Sequence
  MHC Class II binding epitope
YTELYQQLNDLEA

SEQ ID NO: 59 - protein, 13 aa, Artificial Sequence
  MHC Class II binding epitope
TELYQQLNDLEAC

SEQ ID NO: 60 - protein, 13 aa, Artificial Sequence
  MHC Class II binding epitope
ELYQQLNDLEACV

SEQ ID NO: 61 - protein, 13 aa, Artificial Sequence
  MHC Class II binding epitope
YQQLNDLEACVIQ

SEQ ID NO: 62 - protein, 13 aa, Artificial Sequence
  MHC Class II binding epitope
QQLNDLEACVIQG

SEQ ID NO: 63 - protein, 13 aa, Artificial Sequence
  MHC Class II binding epitope
NDLEACVIQGVGV

SEQ ID NO: 64 - protein, 13 aa, Artificial Sequence
  MHC Class II binding epitope
LEACVIQGVGVTE

SEQ ID NO: 65 - protein, 13 aa, Artificial Sequence
  MHC Class II binding epitope
ACVIQGVGVTETP

SEQ ID NO: 66 - protein, 13 aa, Artificial Sequence
  MHC Class II binding epitope
CVIQGVGVTETPL

SEQ ID NO: 67 - protein, 13 aa, Artificial Sequence
  MHC Class II binding epitope
QGVGVTETPLMKE

SEQ ID NO: 68 - protein, 13 aa, Artificial Sequence
  MHC Class II binding epitope
VGVTETPLMKEDS

SEQ ID NO: 69 - protein, 13 aa, Artificial Sequence
  MHC Class II binding epitope
TETPLMKEDSILA

SEQ ID NO: 70 - protein, 13 aa, Artificial Sequence
  MHC Class II binding epitope
TPLMKEDSILAVR

SEQ ID NO: 71 - protein, 13 aa, Artificial Sequence
  MHC Class II binding epitope
PLMKEDSILAVRK

SEQ ID NO: 72 - protein, 13 aa, Artificial Sequence
  MHC Class II binding epitope
KEDSILAVRKYFQ

SEQ ID NO: 73 - protein, 13 aa, Artificial Sequence
  MHC Class II binding epitope
EDSILAVRKYFQR

SEQ ID NO: 74 - protein, 13 aa, Artificial Sequence
  MHC Class II binding epitope
DSILAVRKYFQRI

SEQ ID NO: 75 - protein, 13 aa, Artificial Sequence
  MHC Class II binding epitope
SILAVRKYFQRIT

SEQ ID NO: 76 - protein, 13 aa, Artificial Sequence
  MHC Class II binding epitope
LAVRKYFQRITLY

SEQ ID NO: 77 - protein, 13 aa, Artificial Sequence
  MHC Class II binding epitope
RKYFQRITLYLKE

SEQ ID NO: 78 - protein, 13 aa, Artificial Sequence
  MHC Class II binding epitope
KYFQRITLYLKEK

SEQ ID NO: 79 - protein, 13 aa, Artificial Sequence
  MHC Class II binding epitope
QRITLYLKEKKYS

SEQ ID NO: 80 - protein, 13 aa, Artificial Sequence
  MHC Class II binding epitope
RITLYLKEKKYSP

SEQ ID NO: 81 - protein, 13 aa, Artificial Sequence
  MHC Class II binding epitope
ITLYLKEKKYSPC

SEQ ID NO: 82 - protein, 13 aa, Artificial Sequence
  MHC Class II binding epitope
TLYLKEKKYSPCA

SEQ ID NO: 83 - protein, 13 aa, Artificial Sequence
  MHC Class II binding epitope
LYLKEKKYSPCAW

SEQ ID NO: 84 - protein, 13 aa, Artificial Sequence
  MHC Class II binding epitope
KKYSPCAWEVVRA

SEQ ID NO: 85 - protein, 13 aa, Artificial Sequence
  MHC Class II binding epitope
CAWEVVRAEIMRS

SEQ ID NO: 86 - protein, 13 aa, Artificial Sequence
  MHC Class II binding epitope
WEVVRAEIMRSFS

SEQ ID NO: 87 - protein, 13 aa, Artificial Sequence
  MHC Class II binding epitope
EVVRAEIMRSFSL

SEQ ID NO: 88 - protein, 13 aa, Artificial Sequence
  MHC Class II binding epitope
VRAEIMRSFSLST

SEQ ID NO: 89 - protein, 13 aa, Artificial Sequence
  MHC Class II binding epitope
AEIMRSFSLSTNL

SEQ ID NO: 90 - protein, 13 aa, Artificial Sequence
  MHC Class II binding epitope
EIMRSFSLSTNLQ

SEQ ID NO: 91 - protein, 13 aa, Artificial Sequence
  MHC Class II binding epitope
MRSFSLSTNLQES

SEQ ID NO: 92 - protein, 13 aa, Artificial Sequence
  MHC Class II binding epitope
RSFSLSTNLQESL

SEQ ID NO: 93 - protein, 13 aa, Artificial Sequence
  MHC Class II binding epitope
FSLSTNLQESLRS

SEQ ID NO: 94 - protein, 13 aa, Artificial Sequence
  MHC Class II binding epitope
STKDSSAAWDETL

SEQ ID NO: 95 - protein, 15 aa, Artificial Sequence
  MHC Class II binding epitope
CDLPQTHSLGSRRTL

SEQ ID NO: 96 - protein, 15 aa, Artificial Sequence
  MHC Class II binding epitope
PQTHSLGSRRTLMLL

SEQ ID NO: 97 - protein, 15 aa, Artificial Sequence
  MHC Class II binding epitope
HSLGSRRTLMLLAQM

SEQ ID NO: 98 - protein, 15 aa, Artificial Sequence
  MHC Class II binding epitope
GSRRTLMLLAQMRRI

SEQ ID NO: 99 - protein, 15 aa, Artificial Sequence
  MHC Class II binding epitope
RTLMLLAQMRRISLF

SEQ ID NO: 100 - protein, 15 aa, Artificial Sequence
  MHC Class II binding epitope
MLLAQMRRISLFSCL

SEQ ID NO: 101 - protein, 15 aa, Artificial Sequence
  MHC Class II binding epitope
AQMRRISLFSCLKDR

SEQ ID NO: 102 - protein, 15 aa, Artificial Sequence
  MHC Class II binding epitope
RRISLFSCLKDRHDF

SEQ ID NO: 103 - protein, 15 aa, Artificial Sequence
  MHC Class II binding epitope
SLFSCLKDRHDFGFP

SEQ ID NO: 104 - protein, 15 aa, Artificial Sequence
  MHC Class II binding epitope
SCLKDRHDFGFPQEE

SEQ ID NO: 105 - protein, 15 aa, Artificial Sequence
  MHC Class II binding epitope
KDRHDFGFPQEEFGN

SEQ ID NO: 106 - protein, 15 aa, Artificial Sequence
  MHC Class II binding epitope
HDFGFPQEEFGNQFQ

SEQ ID NO: 107 - protein, 15 aa, Artificial Sequence
  MHC Class II binding epitope
GFPQEEFGNQFQKAE

SEQ ID NO: 108 - protein, 15 aa, Artificial Sequence
  MHC Class II binding epitope
QEEFGNQFQKAETIP

SEQ ID NO: 109 - protein, 15 aa, Artificial Sequence
  MHC Class II binding epitope
FGNQFQKAETIPVLH

SEQ ID NO: 110 - protein, 15 aa, Artificial Sequence
  MHC Class II binding epitope
QFQKAETIPVLHEMI

SEQ ID NO: 111 - protein, 15 aa, Artificial Sequence
  MHC Class II binding epitope
KAETIPVLHEMIQQI

SEQ ID NO: 112 - protein, 15 aa, Artificial Sequence
  MHC Class II binding epitope
TIPVLHEMIQQIFNL

SEQ ID NO: 113 - protein, 15 aa, Artificial Sequence
  MHC Class II binding epitope
VLHEMIQQIFNLFST

SEQ ID NO: 114 - protein, 15 aa, Artificial Sequence
  MHC Class II binding epitope
EMIQQIFNLFSTKDS

SEQ ID NO: 115 - protein, 15 aa, Artificial Sequence
  MHC Class II binding epitope
QQIFNLFSTKDSSAA

SEQ ID NO: 116 - protein, 15 aa, Artificial Sequence
  MHC Class II binding epitope
FNLFSTKDSSAAWDE

SEQ ID NO: 117 - protein, 15 aa, Artificial Sequence
  MHC Class II binding epitope
FSTKDSSAAWDETLL

SEQ ID NO: 118 - protein, 15 aa, Artificial Sequence
  MHC Class II binding epitope
KDSSAAWDETLLDKF

SEQ ID NO: 119 - protein, 15 aa, Artificial Sequence
  MHC Class II binding epitope
SAAWDETLLDKFYTE

SEQ ID NO: 120 - protein, 15 aa, Artificial Sequence
  MHC Class II binding epitope
WDETLLDKFYTELYQ

SEQ ID NO: 121 - protein, 15 aa, Artificial Sequence
  MHC Class II binding epitope
TLLDKFYTELYQQLN

SEQ ID NO: 122 - protein, 15 aa, Artificial Sequence
  MHC Class II binding epitope
DKFYTELYQQLNDLE

SEQ ID NO: 123 - protein, 15 aa, Artificial Sequence
  MHC Class II binding epitope
YTELYQQLNDLEACV

SEQ ID NO: 124 - protein, 15 aa, Artificial Sequence
  MHC Class II binding epitope
LYQQLNDLEACVIQG

SEQ ID NO: 125 - protein, 15 aa, Artificial Sequence
  MHC Class II binding epitope
QLNDLEACVIQGVGV

SEQ ID NO: 126 - protein, 15 aa, Artificial Sequence
  MHC Class II binding epitope
DLEACVIQGVGVTET

SEQ ID NO: 127 - protein, 15 aa, Artificial Sequence
  MHC Class II binding epitope
ACVIQGVGVTETPLM

SEQ ID NO: 128 - protein, 15 aa, Artificial Sequence
  MHC Class II binding epitope
IQGVGVTETPLMKED

SEQ ID NO: 129 - protein, 15 aa, Artificial Sequence
  MHC Class II binding epitope
VGVTETPLMKEDSIL

SEQ ID NO: 130 - protein, 15 aa, Artificial Sequence
  MHC Class II binding epitope
TETPLMKEDSILAVR

SEQ ID NO: 131 - protein, 15 aa, Artificial Sequence
  MHC Class II binding epitope
PLMKEDSILAVRKYF

SEQ ID NO: 132 - protein, 15 aa, Artificial Sequence
  MHC Class II binding epitope
KEDSILAVRKYFQRI

SEQ ID NO: 133 - protein, 15 aa, Artificial Sequence
  MHC Class II binding epitope
SILAVRKYFQRITLY

SEQ ID NO: 134 - protein, 15 aa, Artificial Sequence
  MHC Class II binding epitope
AVRKYFQRITLYLKE

SEQ ID NO: 135 - protein, 15 aa, Artificial Sequence
  MHC Class II binding epitope
KYFQRITLYLKEKKY

SEQ ID NO: 136 - protein, 15 aa, Artificial Sequence
  MHC Class II binding epitope
QRITLYLKEKKYSPC

SEQ ID NO: 137 - protein, 15 aa, Artificial Sequence
  MHC Class II binding epitope
TLYLKEKKYSPCAWE

SEQ ID NO: 138 - protein, 15 aa, Artificial Sequence
  MHC Class II binding epitope
LKEKKYSPCAWEVVR

SEQ ID NO: 139 - protein, 15 aa, Artificial Sequence
  MHC Class II binding epitope
KKYSPCAWEVVRAEI

SEQ ID NO: 140 - protein, 15 aa, Artificial Sequence
  MHC Class II binding epitope
SPCAWEVVRAEIMRS

SEQ ID NO: 141 - protein, 15 aa, Artificial Sequence
  MHC Class II binding epitope
AWEVVRAEIMRSFSL

SEQ ID NO: 142 - protein, 15 aa, Artificial Sequence
  MHC Class II binding epitope
VVRAEIMRSFSLSTN

SEQ ID NO: 143 - protein, 15 aa, Artificial Sequence
  MHC Class II binding epitope
AEIMRSFSLSTNLQE

SEQ ID NO: 144 - protein, 15 aa, Artificial Sequence
  MHC Class II binding epitope
MRSFSLSTNLQESLR

SEQ ID NO: 145 - protein, 15 aa, Artificial Sequence
  MHC Class II binding epitope
FSLSTNLQESLRSKE

SEQ ID NO: 146 - protein, 13 aa, Artificial Sequence
  Antigen
PDYASLRSLVASS

SEQ ID NO: 147 - protein, 13 aa, Artificial Sequence
  Antigen
MQYIKANSKFIGI